(19) United States
(12) Patent Application Publication
(10) Pub. No.: US 2011/0052513 A1
(43) Pub. Date: Mar. 3, 2011

(54) CATIONIC SURFACTANT COMPOUNDS, USE THEREOF AS CONDITIONER, COSMETIC TREATMENT METHOD, AND COSMETIC OR PHARMACEUTICAL COMPOSITIONS COMPRISING SAME

(75) Inventors: Laure Ramos-Stanbury, Sceaux (FR); Herve Andrean, Paris (FR); Christian Blaise, Saint Mande (FR)

(73) Assignee: L' Oreal, Paris (FR)

(21) Appl. No.: 12/865,829

(22) PCT Filed: Feb. 3, 2009
(86) PCT No.: PCT/FR09/50163

§ 371 Date: Nov. 12, 2010

Related U.S. Application Data
(60) Provisional application No. 61/027,836, filed on Feb. 12, 2008.

(30) Foreign Application Priority Data
Feb. 4, 2008 (FR) 0850662

(51) Int. Cl. A61K 31/215; C07C 229/28; C07C 229/32; C07C 237/18; A61K 31/16; A61K 8/42; A61K 8/37; A61Q 17/04; A61Q 5/06; A61Q 5/12; A61Q 19/00; A61Q 1/00; A61P 17/00; A61P 17/18

(52) U.S. Cl. 424/59; 560/118; 560/124; 560/121; 560/116; 564/189; 514/530; 514/531; 514/613; 424/70.1; 424/70.28

(57) ABSTRACT

The present patent application relates to novel surface-active cationic compounds, to their use, in particular as hair conditioning agent, to a cosmetic treatment method, in particular for the hair, employing the said compounds, and to the cosmetic or pharmaceutical compositions, in particular hair compositions, comprising the said surfactants.

The present invention relates to novel cationic compounds, to the cosmetic compositions comprising them and to their use, in particular for cosmetically treating the hair.

It is well known that hair which has been sensitized, in particular damaged and/or embrittled, to various degrees under the action of atmospheric agents or under the action of mechanical and/or chemical treatments, such as dyeing operations, bleaching operations or perming operations, is often difficult to disentangle and to style and also lacks softness.

Cosmetic compositions comprising conditioning cationic surfactants, such as those described in US 2006/0078529, have already been proposed for the treatment of keratinous substances and in particular the hair. However, such compositions still do not have the desired cosmetic qualities, in particular in terms of sensorial properties, in particular of feel of the hair after treatment.

In point of fact, the Applicant Company has now discovered, unexpectedly and surprisingly, that some very specific cationic compounds can contribute advantageous conditioning properties to the hair, in particular relating to the improvement in the suppleness of the hair when the composition is applied to wet hair and then the achievement, after rinsing, of hair still exhibiting an improved suppleness, the hair thus being less stiff. This rendering of the hair supple is particularly noteworthy with the compositions according to the invention.

Furthermore, in addition to the suppleness, these compositions also make it possible to improve the disentangling, the smoothing, the ability to be combed and the manageability of the hair; the shaping of the hair is easier and the feel of the hair is very pleasant and fluid.

In addition, the compounds according to the invention can be easily conveyed in aqueous cosmetic media, which facilitates the use thereof.

Thus, the subject-matter of the present invention is a compound of formula (I) or (II) as defined below.

Another subject-matter of the invention is a cosmetic or pharmaceutical composition comprising, in a physiologically acceptable medium, at least one compound of formula (I) or (II) as defined below.

Another subject-matter of the invention is the use of at least one compound of formula (I) or (II) as defined below, or of a composition as defined below, as hair conditioning agent.

The compounds according to the invention thus correspond to the formula (I) or (II):

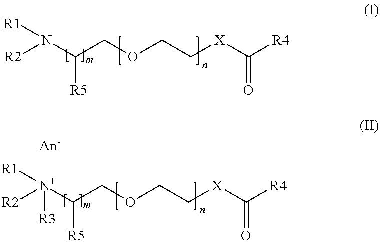

in which:

m is an integer between 1 and 10;

n is an integer between 0 and 10;

X represents O, NH or S;

R1, R2 and R3 denote, independently of one another, a linear C₁-C₂₂ or branched C₃-C₂₈ alkyl group or a linear C₂-C₂₂ or branched C₃-C₂₈ alkenyl group, these groups being optionally substituted by one or more identical or different radicals chosen from hydroxyl (—OH) and amino (—NRR′) radicals, with R and R′ chosen, independently of one another, from H and C₁-C₆ alkyl; or else

R1 and R2 form, with the nitrogen atom to which they are connected, a saturated or unsaturated carbon-comprising heterocycle comprising 5 or 6 ring members, it being possible for one or two nonadjacent carbon atoms optionally to be replaced by an oxygen, nitrogen (—NR″), with R″=H or C₁-C₂₂ alkyl, or sulphur atom; it being possible for the said heterocycle optionally to be substituted by one or more identical or different radicals chosen from aryl, C₁-C₂₂ alkyl, hydroxyl or amino (—NRR′) radicals, with R and R′ chosen, independently of one another, from H and C₁-C₆ alkyl; and R3 has the above definition, if appropriate;
R4 denotes:

either a linear or branched C₄-C₂₂ alkyl radical (saturated) comprising, as interruption in the chain, from one to three identical or different and saturated or unsaturated carbon-comprising rings each comprising 3, 4, 5 or 6 ring members;

the said ring(s) being optionally substituted by an aryl, itself optionally substituted by one or more identical or different radicals chosen from C₁-C₆ alkyl, hydroxyl (—OH) and amino (—NRR′) radicals, with R and R′ chosen, independently of one another, from H and C₁-C₆ alkyl;
it being understood that, when R4 denotes an alkyl radical including 2 or 3 carbon-comprising rings, the said rings are separated from one another by a divalent methylene —CH₂— or ethylene radical;

or a linear or branched C₄-C₂₂ alkyl or alkenyl radical (saturated or unsaturated) comprising, at the chain end, a saturated or unsaturated and non-aromatic carbon-comprising ring comprising 5 or 6 ring members;

R5 denotes a hydrogen atom or an OR or NRR′ radical with R and R′ chosen, independently of one another, from H and C₁-C₆ alkyl; it being understood that, when m is greater than or equal to 2, the R5 radicals are identical or different;

An⁻ denotes an organic or inorganic anion or a mixture of organic or inorganic anions, in order to ensure the electrical neutrality of the compounds of formula (II).

Preferably, m is an integer between 1 and 4, in particular 1, 2 or 3, preferably 1 or 2.

Preferably, n is an integer between 0 and 4, in particular 0, 1, 2 or 3, preferably 0 or 1.

Preferably, X represents O or NH.

Preferably, R1 denotes a linear C₁-C₄ alkyl or branched C₃-C₄ alkyl group or a linear C₂-C₄ alkenyl or branched C₃-C₄ alkenyl group. Preferably, R1 represents methyl or ethyl.

Preferably, R2 denotes a linear C₁-C₄ alkyl or branched C₃-C₄ alkyl group or a linear C₂-C₄ alkenyl or branched C₃-C₄ alkenyl group. Preferably, R2 represents methyl or ethyl.

Preferably, R3 denotes a linear C₁-C₂₂ alkyl or branched C₃-C₂₂ alkyl group or a linear C₂-C₂₂ alkenyl or branched C₃-C₂₂ alkenyl group. Preferably, R3 represents a linear C₁-C₁₈ alkyl group.

Preferably, R4 denotes:

either a linear C₄-C₂₂ alkyl radical comprising, as interruption in the chain, from one to three identical or different and saturated or unsaturated carbon-comprising rings each comprising 3, 4, 5 or 6 ring members; preferably a linear C₁₂-C₂₁ alkyl radical comprising, as interruption in the chain, from one to three identical and saturated carbon-comprising rings each comprising 3, 4, 5 or 6 ring members; the said alkyl radical very particularly comprising, as interruption, from one to three cyclopropyl rings, it being understood that, when 2 or 3 rings are present, they are separated from one another by a divalent methylene —CH₂— radical;

In particular, R4 can be a linear or branched C₄-C₂₂ alkyl radical comprising, as interruption in the chain, one of the following divalent sequences:

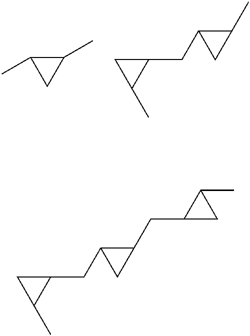

or a linear or branched C₄-C₂₁ alkyl or alkenyl radical (saturated or unsaturated) comprising, at the chain end, a saturated or unsaturated and non-aromatic carbon-comprising ring comprising 5 ring members.

Preferably, R5 denotes a hydrogen atom or a hydroxyl (OH) radical.

Preferably, An⁻ denotes an anion or a mixture of anions chosen from acetate, lactate, tartrate, citrate, halide, SO₄²⁻, HSO₄⁻, MeSO₄⁻, EtSO₄⁻, mesylate and tosylate and very particularly Cl⁻, Br⁻, MeSO₄⁻, EtSO₄⁻, mesylate and tosylate; and their mixtures.

The compounds of formula (II) can be used as is or in the form of solvates, in particular of hydrates.

The compounds of formula (I) or (II) can be used alone or as a mixture.

Mention may be made, among the preferred compounds of formula (I) or (II), of the following compounds and their salts or solvates, alone or as a mixture:

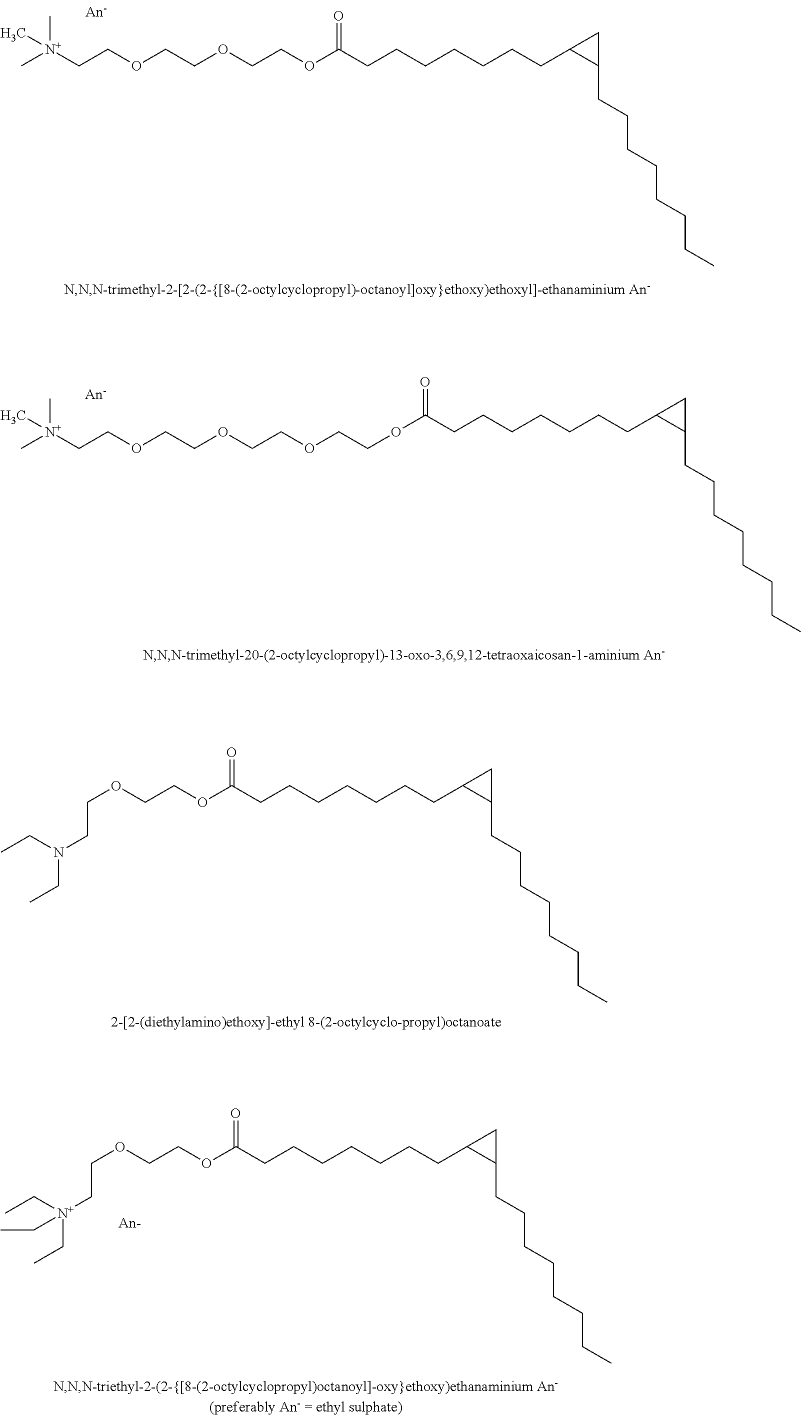

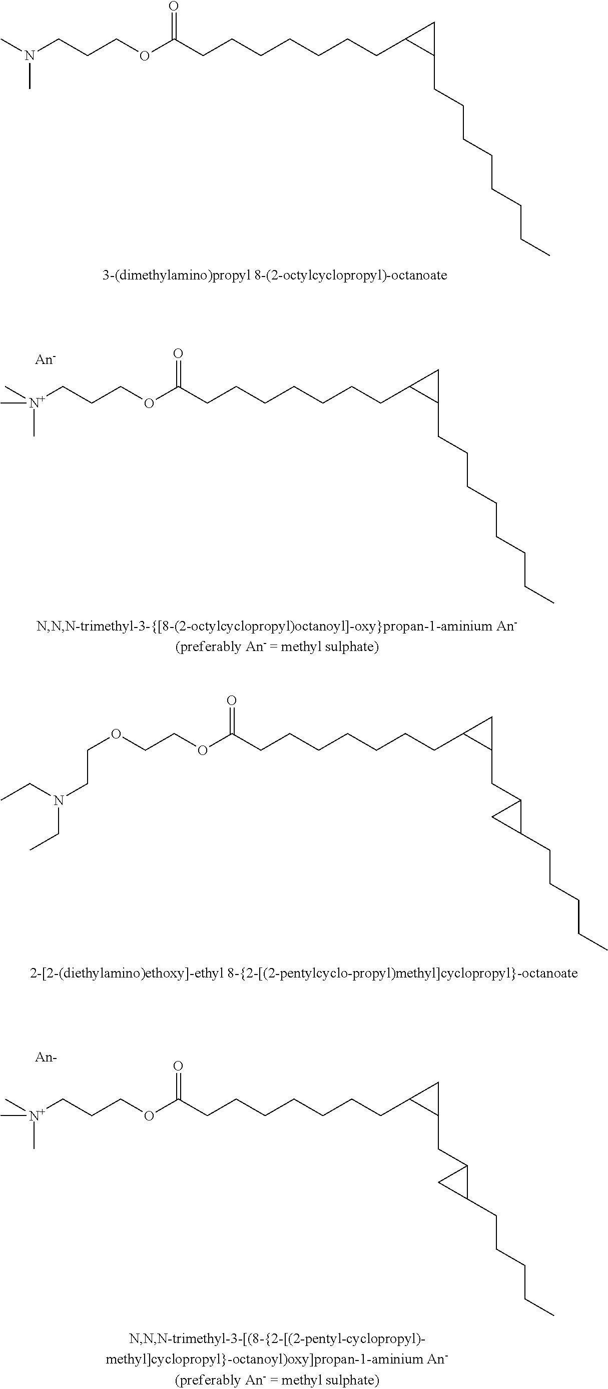

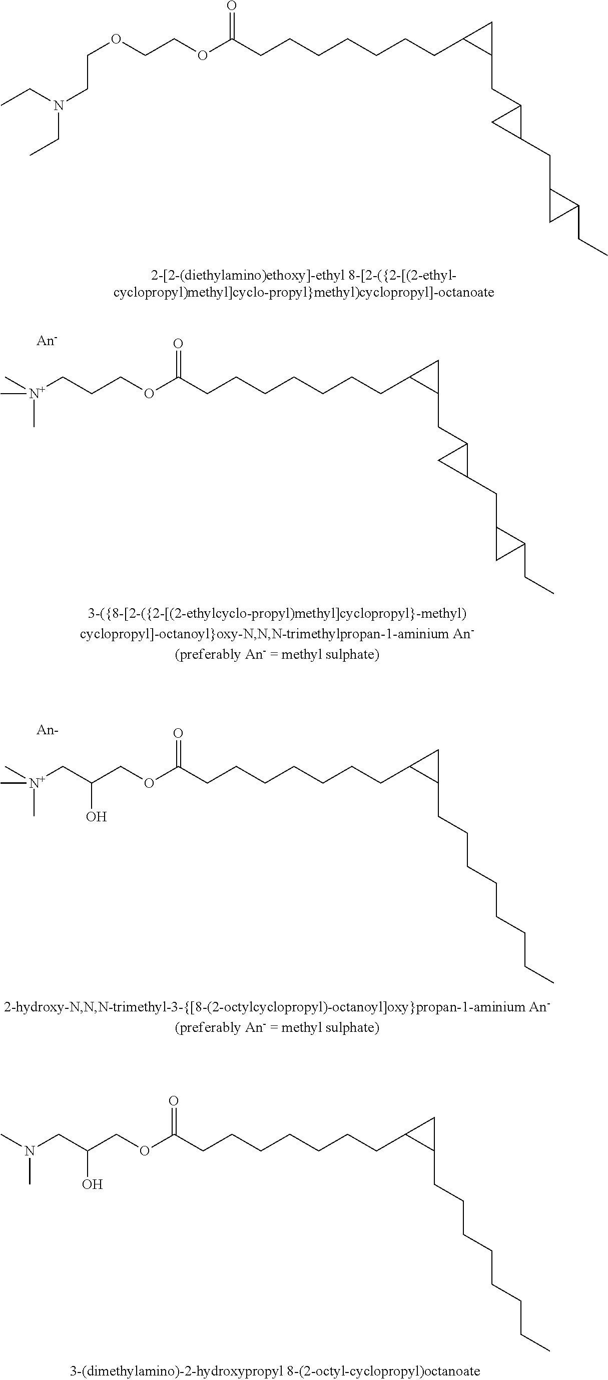

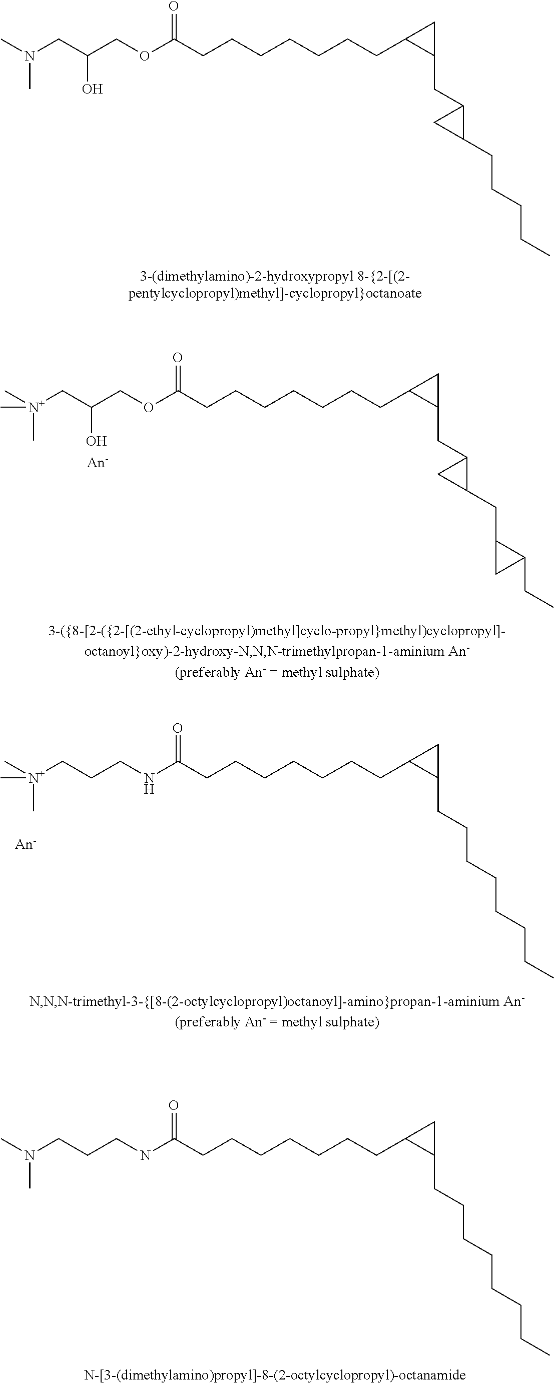

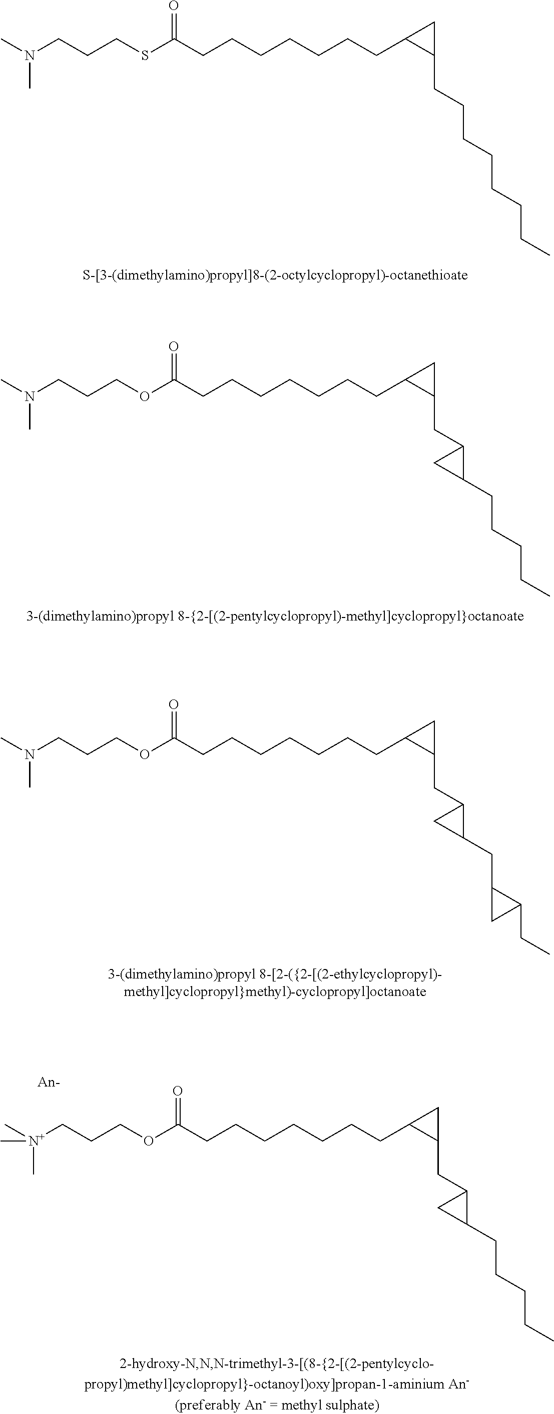

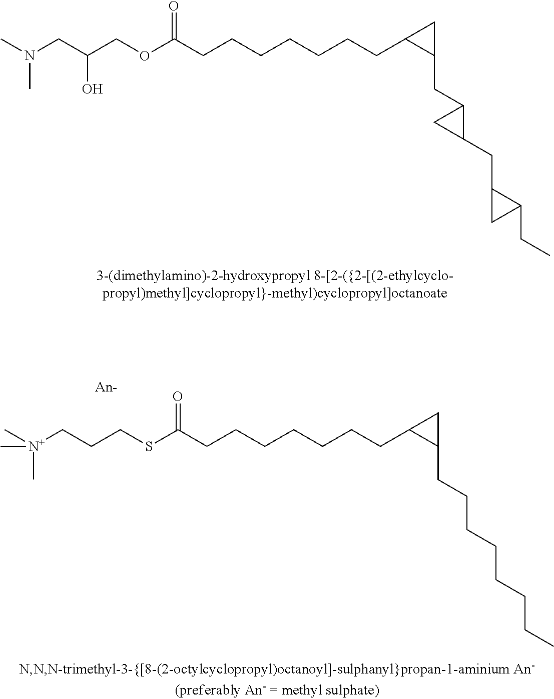

Other preferred compounds of formula (I) or (II) are the following compounds, and their salts or solvates, it being understood that these compounds can be employed alone or as a mixture:

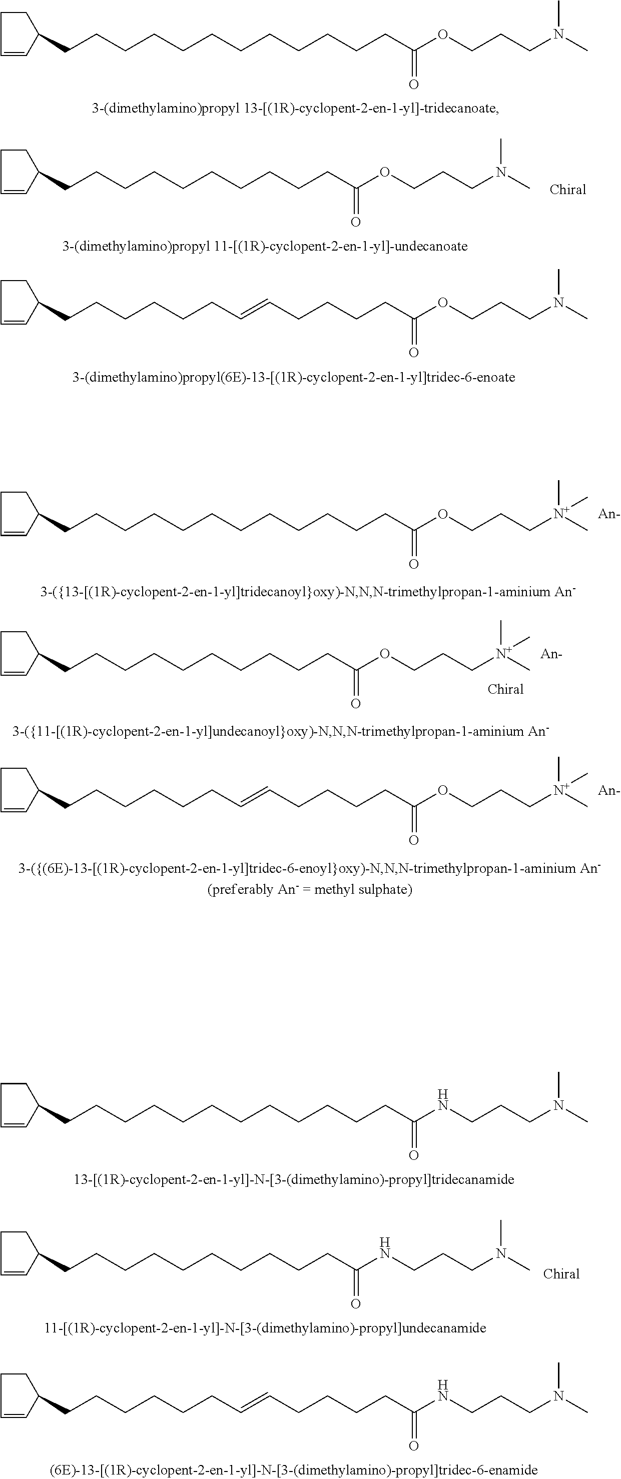

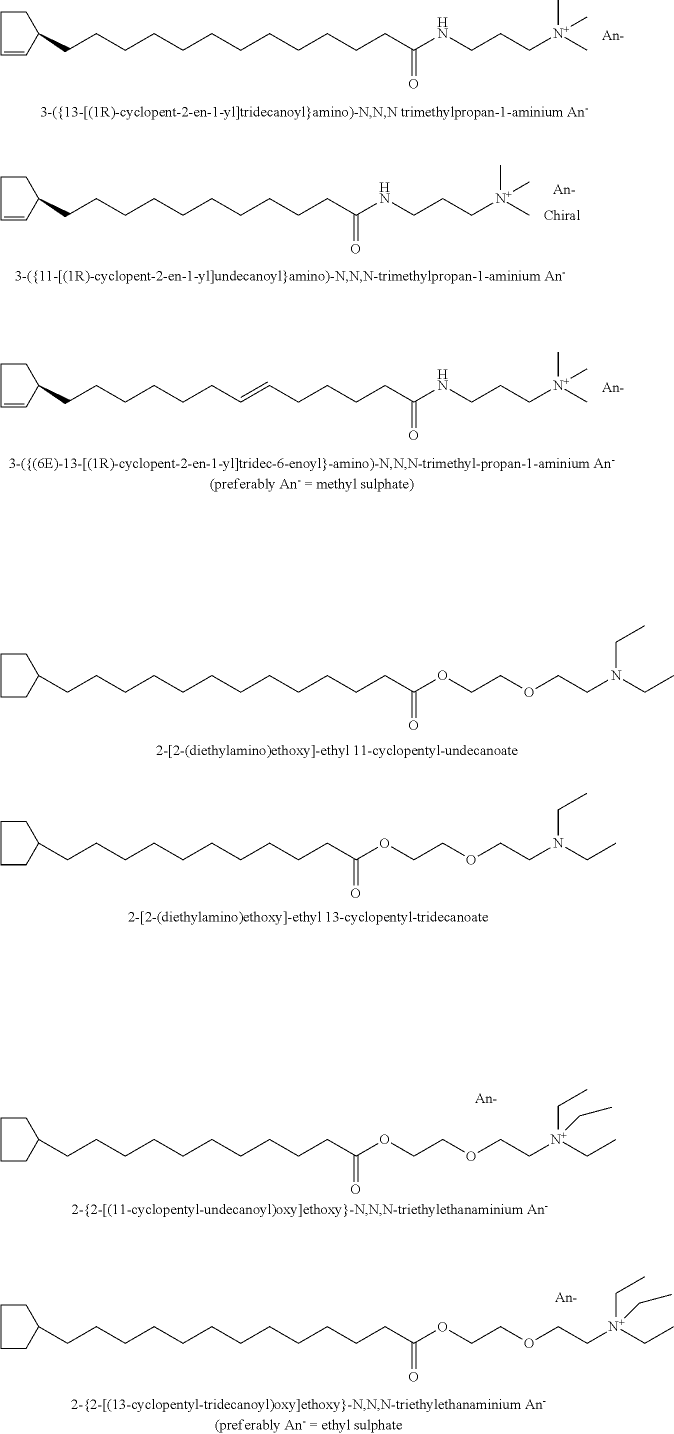

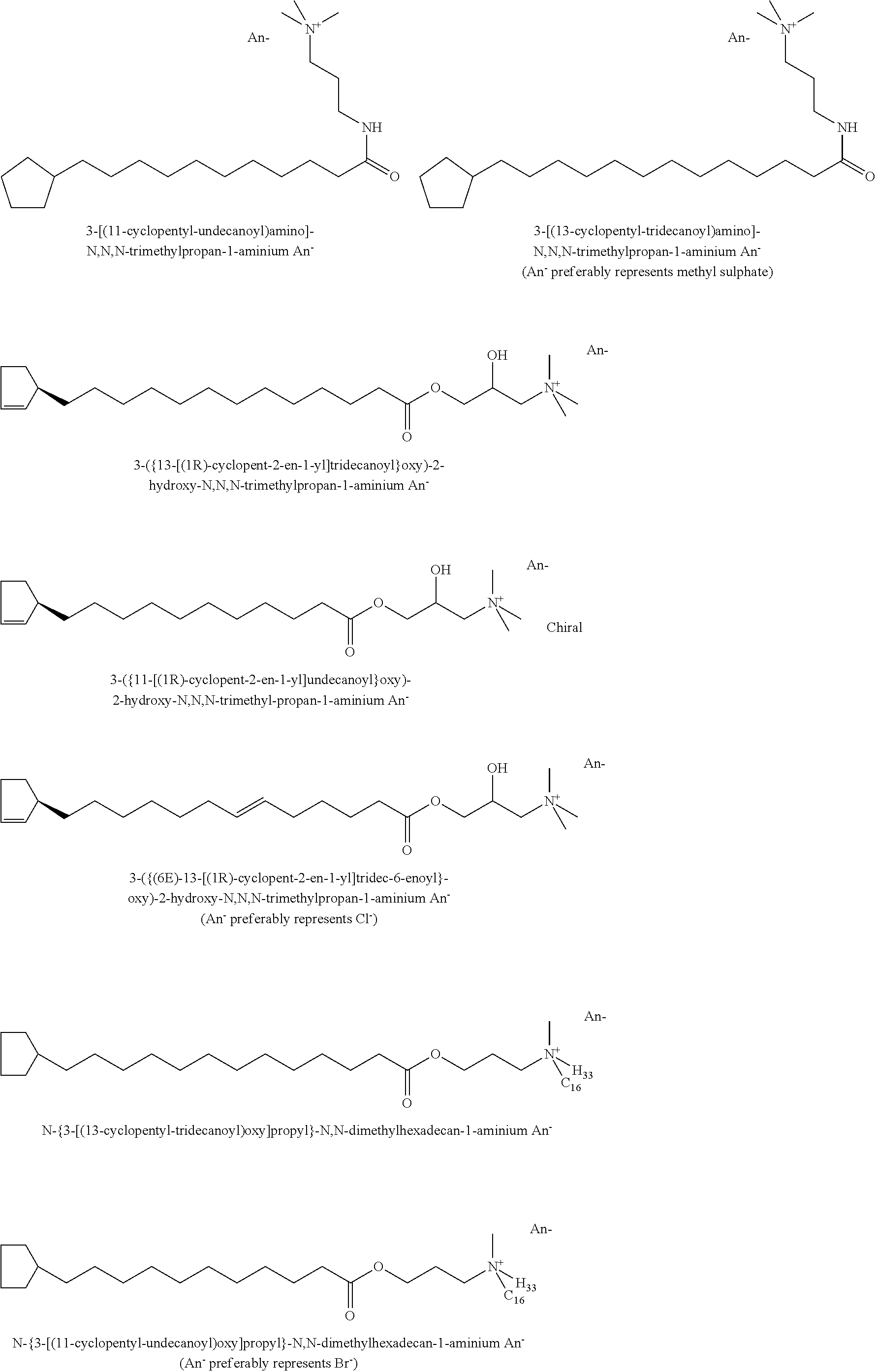

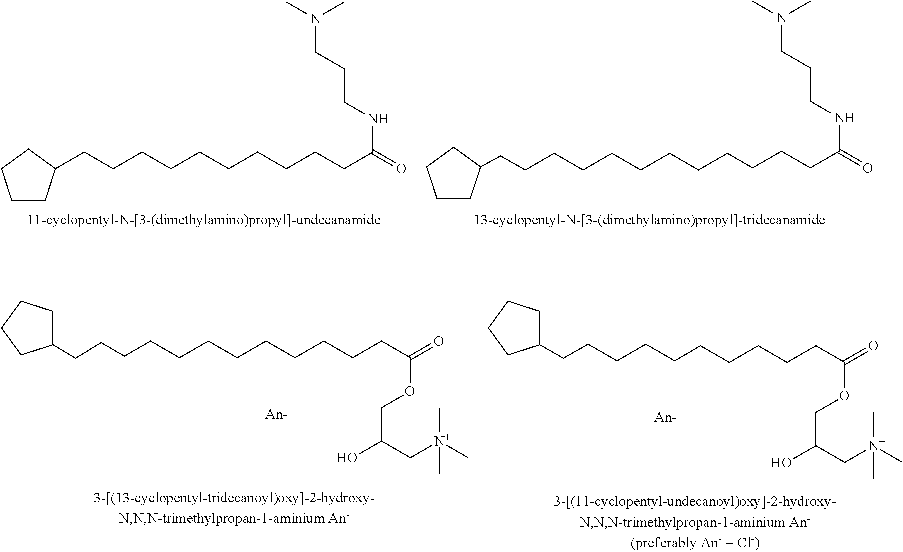

In a preferred embodiment, it is possible to employ a mixture of compounds according to the invention and in particular the following mixtures:

mixture of 3-(dimethylamino)propyl 13-[(1R)-cyclopent-2-en-1-yl]tridecanoate+3-(dimethylamino)-propyl 11-[(1R)-cyclopent-2-en-1-yl]undecanoate+3-(dimethylamino)propyl (6E)-13-[(1R)-cyclopent-2-en-1-yl]tridec-6-enoate, in particular in the proportions 40/55/5;

mixture of 13-[(1R)-cyclopent-2-en-1-yl]-N-[3-(dimethylamino)propyl]tridecanamide+11-[(1R)-cyclopent-2-en-1-yl]-N-[3-(dimethylamino)propyl]-undecanamide+(6E)-13-[(1R)-cyclopent-2-en-1-yl]-N-[3-(dimethylamino)propyl]tridec-6-enamide, in particular in the proportions 40/55/5;

mixture of 2-[2-(diethylamino)ethoxy]ethyl 11-cyclopentylundecanoate and 2-[2-(diethylamino)ethoxy]-ethyl 13-cyclopentyltridecanoate, in particular in the proportions 50/50;

mixture of 3-[(11-cyclopentylundecanoyl)amino]-N,N,N-trimethylpropan-1-aminium An⁻ and 3-[(13-cyclo-pentyltridecanoyl)amino]-N,N,N-trimethylpropan-1-aminium An⁻, in particular in the proportions 50/50; An⁻ preferably represents methyl sulphate;

mixture of N-{3-[(13-cyclopentyltridecanoyl)-oxy]propyl}-N,N-dimethyl hexadecan-1-aminium An⁻ and N-{3-[(11-cyclopentylundecanoyl)oxy]propyl}-N,N-dimethyl-hexadecan-1-aminium An⁻; An⁻ preferably represents bromide; in particular in the proportion 50/50;

mixture of 3-[(13-cyclopentyltridecanoyl)oxy]-2-hydroxy-N,N,N-trimethylpropan-1-aminium An⁻ and 3-[(11-cyclopentylundecanoyl)oxy]-2-hydroxy-N,N,N-trimethyl-propan-1-aminium An⁻; An⁻ preferably represents chloride; in particular in the proportions 50/50;

mixture of 3-({13-[(1R)-cyclopent-2-en-1-yl]-tridecanoyl}oxy)-N,N,N-trimethylpropan-1-aminium An⁻+3-({11-[(1R)-cyclopent-2-en-1-yl]undecanoyl}oxy)-N,N,N-trimethylpropan-1-aminium An⁻+3-({(6E)-13-[(1R)-cyclopent-2-en-1-yl]tridec-6-enoyl}oxy)-N,N,N-trimethylpropan-1-aminium An⁻; in particular in the proportions 40/55/5; An⁻ preferably represents methyl sulphate;

mixture of 3-({13-[(1R)-cyclopent-2-en-1-yl]-tridecanoyl}amino)-N,N,N-trimethylpropan-1-aminium An⁻+3-({11-[(1R)-cyclopent-2-en-1-yl]undecanoyl}amino)-N,N,N-trimethylpropan-1-aminium An⁻+3-({(6E)-13-[(1R)-cyclopent-2-en-1-yl]tridec-6-enoyl}amino)-N,N,N-trimethylpropan-1-aminium An⁻, in particular in the proportions 40/55/5; An⁻ preferably represents methyl sulphate;

mixture of 2-{2-[(11-cyclopentylundecanoyl)-oxy]ethoxy}-N,N,N-triethylethanaminium An⁻+2-{2-[(13-cyclopentyltridecanoyl)oxy]ethoxy}-N,N,N-triethyl-ethanaminium An⁻; An⁻ preferably represents ethyl sulphate; in particular in the proportions 50/50;

mixture of 3-({13-[(1R)-cyclopent-2-en-1-yl]-tridecanoyl}oxy)-2-hydroxy-N,N,N-trimethylpropan-1-aminium An⁻+3-({11-[(1R)-cyclopent-2-en-1-yl]-undecanoyl}oxy)-2-hydroxy-N,N,N-trimethylpropan-1-aminium An⁻+3-({(6E)-13-[(1R)-cyclopent-2-en-1-yl]tridec-6-enoyl}oxy)-2-hydroxy-N,N,N-trimethylpropan-1-aminium An⁻; An⁻ preferably represents chloride;

mixture of 11-cyclopentyl-N-[3-(dimethylamino)-propyl]undecanamide and 13-cyclopentyl-N-[3-(dimethyl-amino)propyl]tridecanamide; in particular in the proportions 50/50.

The compounds (I) and (II) according to the invention can be prepared by a person skilled in the art on the basis of his general knowledge.

The synthesis of some compounds (I) can in particular be carried out according to the process described in Patent FR 2 869 902.

Some compounds (II) can be prepared according to the following synthesis scheme:

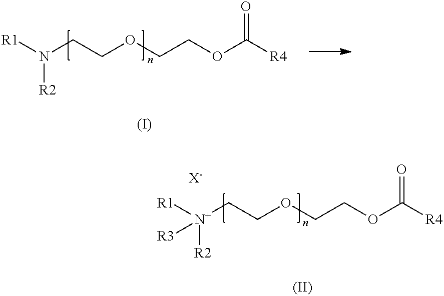

The quaternized ester (II) can be obtained by reacting the amine (I) with an alkylating agent, such as, for example, methyl iodide, dimethyl sulphate, ethyl iodide, diethyl sulphate or a haloalkane, such as a bromohexadecane. The tertiary amine (I) and the alkylating agent can be mixed and heated at 15° C.-140° C. for 2 to 80 hours. After cooling, the excess alkylating agent can be removed by washing operations with diisopropyl ether. The solid obtained can be filtered off, preferably under an inert atmosphere, washed and dried under reduced pressure, optionally in the presence of P₂O₅.

An ion exchange can be carried out on conclusion of the reaction, by contact with an ion-exchange resin chosen according to the exchanges desired. These resins are, for example, IRA 402 (alkyl sulphates to chlorides exchange) or IRA 400 (iodide to chloride exchange). Thus, the anion can be exchanged by treatment of an aqueous or (aqueous) alcoholic solution of the compound with the ion-exchange resin. The solvent can be removed and the product can be washed, for example with diisopropyl ether, and then filtered off and dried under reduced pressure, optionally in the presence of P₂O₅.

The compounds of formula (I) or (II) in which R4 denotes a linear or branched C₄-C₂₂ alkyl or alkenyl radical (saturated or unsaturated) comprising, at the chain end, a saturated or unsaturated and non-aromatic carbon-comprising ring comprising 5 ring members can be prepared by saponification, followed by activation of the ester and condensation, starting from chaulmoogra oil.

Chaulmoogra oil is an essential oil extracted from the seeds of woody plants of the tropical regions belonging to the family of the Flacourtiaceae, in particular of a tree of type such as Hydnocarpus wightiana and Taraktogenos kurzii. These plants are essentially of Asian origin, in particular from India, Vietnam and the Philippines, and also from central Africa and South America, in particular from Brazil.

The seeds from which the chaulmoogra oil is extracted comprise a high proportion of lipids, generally of between 30 and 50%, depending on the species, 15 to 20% of proteins and their hydrolysis products, and 4 to 6% of mineral matter, and also from 1 to 3% of non-saponifiable substances, glycerides of unsaturated fatty acids comprising a pentene ring, essentially composed of chaulmoogric acid, hydnocarpic acid and gorlic acid, of formula:

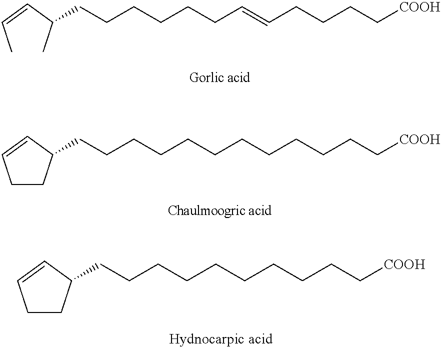

The contents of each of these three acids depend on the origin of the species. Chaulmoogra oil is also found to comprise fatty acids of the palmitic, oleic, palmitoleic, stearic and myristic type and traces of alepric acid and aleprilic acid.

The chaulmoogra oil used in the compositions of the present invention can be extracted from seeds of plants of varieties such as:

Hydnocarpus wightiana,

Taraktogenos kurzii,

Hydnocarpus alpina,

Hydnocarpus anthelmintica,

Hydnocarpus cauliflora,

Hydnocarpus dawnensis,

Hydnocarpus heterophylla,

Hydnocarpus hutchinsonii,

Hydnocarpus ovoidea,

Hydnocarpus subfalcata,

Hydnocarpus venenata,

Hydnocarpus verrucosa,

Hydnocarpus woodii,

Hydnocarpus calvipetala,

Hydnocarpus ilicifolia,

Hydnocarpus octandra,

Gynocardia odorata,

Oncoba echinata,

Caloncoba glauca,

Caloncoba welwitschii,

Carpotroche brasiliensis,

Carpotroche amazonica,

Asteriastigma macrocarpa,

Mayna odorata and Lindackeria dentata.

The three main acids present in chaulmoogra oil can be hydrogenated under standard catalytic hydrogenation conditions in order to result in the corresponding completely hydrogenated acids, which correspond to some compounds of formula (I) according to the present invention:

11-cyclopentylundecanoic acid of formula:

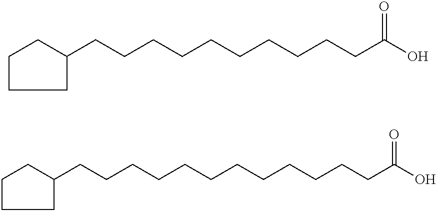

13-cyclopentyltridecanoic acid of formula:

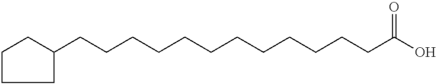

The compounds according to the invention, (I) or (II), have a very particular application in the cosmetic or pharmaceutical field, in particular in the hair field, especially as conditioning agent.

The amount of compound (I) or (II), alone or as a mixture, present in the compositions depends, of course, on the type of composition and on the properties desired and can vary within a very broad range generally of between 0.01 and 50% by weight, preferably between 0.1 and 30% by weight, in particular between 0.5 and 25% by weight, indeed even between 1 and 20% by weight, better still between 1.5 and 10% by weight, with respect to the total weight of the composition.

The compositions according to the invention can very obviously comprise a mixture of compounds of formula (I) or (II).

The compositions according to the invention can be provided in all the formulation forms conventionally used and in particular in the form of an aqueous, alcoholic, aqueous/alcoholic or oily solution or suspension, of a solution or dispersion of the lotion or serum type, of an emulsion, in particular with a liquid or semiliquid consistency, of the O/W, W/O or multiple type, of a suspension or emulsion with a soft consistency of (O/W) or (W/O) cream type, of an aqueous or anhydrous gel, or of any other cosmetic form.

These compositions can be packaged in pump-action sprays or in aerosol containers, in order to provide for application of the composition in the vaporized form (lacquer) or in the foam form. Such packaging forms are indicated, for example, when it is desired to obtain a spray or a foam, for the treatment of the hair. In these cases, the composition preferably comprises at least one propellant.

Preferably, the composition is provided in the form of an emulsion comprising the compound of formula (I) or (Ia) in dispersion in an aqueous phase or else in solution in a fatty phase.

The compositions according to the invention comprise a physiologically acceptable medium, that is to say a medium compatible with keratinous substances, in particular the skin of the face or of the body, the lips, the hair, the eyelashes, the eyebrows and the nails. The said physiologically acceptable medium is preferably a cosmetically acceptable medium, the composition then being a cosmetic composition intended in particular for a topical application.

The said physiologically acceptable medium preferably comprises at least one standard cosmetic ingredient chosen in particular from C₁-C₄₀ alcohols, carbon-comprising oils, water, C₈-C₄₀ esters, C₈-C₄₀ acids, nonionic surfactants, cationic surfactants, anionic surfactants, amphoteric surfactants, zwitterionic surfactants, propellants, sunscreens, moisturizing agents, antidandruff agents, antioxidants, reducing agents, oxidation bases, couplers, oxidizing agents, direct dyes, hair-straightening agents, pearlescent and opacifying agents, plasticizing or coalescence agents, hydroxy acids, pigments, fillers, silicones, organic solvents, polymeric or nonpolymeric thickeners, emulsifiers or polymers, in particular conditioning or styling polymers. The said medium can very obviously comprise several cosmetic ingredients appearing in the above list.

According to their nature and the destination of the composition, the standard cosmetic ingredients can be present in standard amounts which can be easily determined by a person skilled in the art and which can be, for each ingredient, between 0.01 and 80% by weight.

The composition can in particular comprise water and/or one or more C₁-C₄₀ alcohols; mention may in particular be made of C₁-C₇ aliphatic or aromatic mono-alcohols, polyols and polyol ethers, which can thus be employed alone or as a mixture with water; advantageously, the composition comprises a water/ethanol, water/isopropanol or water/benzyl alcohol mixture.

The carbon-comprising oils, in particular hydrocarbon oils, and/or the silicone oils can be present in a proportion of 0.01 to 20% by weight, in particular of 0.02 to 10% by weight, with respect to the total weight of the composition. Mention may in particular be made of vegetable, animal or mineral oils, which are or are not hydrogenated, saturated or unsaturated, linear or branched and cyclic or aliphatic hydrocarbon synthetic oils, such as, for example, poly-α-olefins, in particular polydecenes and polyisobutenes, volatile or nonvolatile, organomodified or nonorganomodified and water-soluble or water-insoluble silicone oils, fluorinated or perfluorinated oils, and their mixtures.

The alcohols, the esters and the acids having from 8 to 40 carbon atoms can be present in a proportion of 0.01 to 50% by weight, in particular of 0.1 to 20% by weight, with respect to the total weight of the composition.

Mention may in particular be made of fatty alcohols possessing linear or branched C₁₂-C₃₂, in particular C₁₂-C₂₆, chains and especially of cetyl alcohol, stearyl alcohol, cetearyl alcohol, isostearyl alcohol, octyldodecanol, 2-butyloctanol, 2-hexyldecanol, 2-undecylpentadecanol, oleyl alcohol or linoleyl alcohol.

Mention may also be made of oxyalkylenated, in particular oxyethylenated, C₈-C₄₀, in particular C₁₆-C₂₀, fatty alcohols, preferably comprising from 10 to 50 mol of ethylene oxide and/or propylene oxide, such as oleth-12, ceteareth-12 and ceteareth-20, oxypropyl-enated stearyl alcohol comprising in particular 15 mol of propylene oxide, oxyethylenated lauryl alcohol comprising in particular more than 7 oxyethylene groups, and their mixtures.

Mention may also be made of fatty acids possessing linear or branched C₁₆-C₄₀ chains and in particular of 18-methyleicosanoic acid, coconut oil or hydrogenated coconut oil acids, stearic acid, lauric acid, palmitic acid, oleic acid, behenic acid and their mixtures.

Mention may also be made of fatty esters possessing linear or branched chains and comprising in total from 8 to 40 carbon atoms, such as esters of monoalcohols or of polyols of fatty acids comprising from 8 to 30 carbon atoms, and their oxyalkylenated and in particular oxyethylenated derivatives, the polyols being preferably chosen from sugars, C₂-C₆ alkylene glycols, glycerol, polyglycerols, sorbitol, sorbitan, polyethylene glycols, polypropylene glycols and their mixtures. Mention may be made, as esters of monoalcohols, of isopropyl myristate or palmitate, and also myristyl, cetyl and stearyl myristates, palmitates and stearates, alone or as a mixture.

The composition can also comprise, as carbon-comprising oils, vegetable oils, such as avocado oil, olive oil, apricot oil, argan oil, jojoba oil or shea butter, which can be present in a proportion of 0.1 to 10% by weight in the composition, in particular of 0.2 to 5% by weight.

In a preferred embodiment, the composition according to the invention comprises C₈-C₄₀ fatty alcohols which can be present very particularly in an amount of 1 to 15% by weight, in particular of 2.5 to 10% by weight, indeed even of 3 to 8% by weight, in the cosmetic composition.

These fatty alcohols can be saturated or unsaturated and linear or branched; mention may in particular be made of cetyl alcohol, stearyl alcohol, isostearyl alcohol, oleyl alcohol, ricinoleyl alcohol or linoleyl alcohol, alone or as a mixture; saturated fatty alcohols, alone or as a mixture, are preferred.

The nonionic, cationic, anionic, amphoteric or zwitterionic surfactants, other than those of formula (I), and their mixtures can be present in a proportion of 0.01 to 50% by weight, in particular of 0.1 to 40% by weight, indeed even of 0.5 to 30% by weight, better still of 1 to 15% by weight, with respect to the total weight of the composition.

In particular, the ratio by weight of the amount of fatty alcohols to the amount of surfactants in the composition is preferably greater than or equal to 1.5, in particular between 1.5 and 10, indeed even between 1.6 and 8, better still between 1.7 and 6.

The propellants can be present in a proportion of 5 to 90% by weight, with respect to the total weight of the composition, more particularly in a proportion of 10 to 60% by weight.

The sunscreens can be present in a proportion of 0.01 to 20% by weight, in particular of 0.5 to 10% by weight, with respect to the total weight of the composition.

The moisturizing agents can be present in a proportion of 0.01 to 20% by weight, in particular of 0.1 to 7% by weight, with respect to the total weight of the composition.

The antidandruff agents can be present in a proportion of 0.001 to 20% by weight, in particular of 0.01 to 10% by weight, with respect to the total weight of the composition, preferably of 0.1 to 5% by weight.

The antioxidants can be present in a proportion of 0.05 to 1.5% by weight, with respect to the total weight of the composition.

The reducing agents can be present in a proportion of 0.1 to 30% by weight, in particular of 0.5 to 20% by weight, with respect to the total weight of the composition.

The oxidation bases can be present in an amount of between 0.001 and 10% by weight, preferably of 0.005 to 6% by weight, of the total weight of the composition.

The couplers can be present in an amount of between 0.001 and 10% by weight, preferably of 0.005 to 6% by weight, of the total weight of the composition.

The oxidizing agents can be present in an amount of between 1 and 40% by weight, preferably of between 1 and 20% by weight, with respect to the weight of the composition.

The direct dyes can be present in an amount of between 0.001 and 20% by weight, preferably of 0.01 to 10% by weight, with respect to the total weight of the composition.

The hair-straightening agents can be present in a proportion of 0.01 to 3.5% by weight, in particular of 0.05 to 1.5% by weight, with respect to the total weight of the composition.

The pearlescent and opacifying agents can be present in a proportion of 0.01 to 3% by weight, in particular of 0.05 to 2.5% by weight, with respect to the total weight of the composition.

The plasticizing or coalescence agents can be present in a proportion of 0.1 to 25% by weight, in particular of 1 to 10% by weight, with respect to the total weight of the composition.

The hydroxy acids can be present in a proportion of 1 to 10% by weight, in particular of 2 to 5% by weight, with respect to the total weight of the composition.

The pigments and fillers can be present in a proportion of 0.01 to 50% by weight, in particular of 0.02 to 30% by weight, with respect to the total weight of the composition.

The silicones can be volatile or nonvolatile; mention may in particular be made of polyorganosiloxanes which are or are not modified, namely polyorganosiloxane oils, gums and resins, as is or in the form of solutions in organic solvents or in the form of emulsions or microemulsions. They can be present in an amount of 0.01 to 40% by weight, in particular of 0.05 to 20% by weight, with respect to the total weight of the composition.

The thickeners can be present in a proportion of 0.01 to 10% by weight, in particular of 0.1 to 5% by weight, with respect to the total weight of the composition.

The polymers, in particular polymers which are soluble in water or soluble in carbon-comprising and/or silicone oils, can be present in a proportion of 0.01 to 20% by weight, in particular of 0.1 to 10% by weight, with respect to the total weight of the composition.

A person skilled in the art will take care to choose the ingredients participating in the composition, and their amounts, so that they do not interfere with the properties of the compositions of the present invention.

The cosmetic composition according to the invention can be provided in the form of a product for caring for, cleaning and/or making up the skin of the body or of the face, the lips, the eyebrows, the eyelashes, the nails and the hair, of an antisun or self-tanning product, of a body hygiene product or of a hair product, in particular for caring for, cleaning, styling, shaping or dyeing the hair.

In particular, it has a particularly advantageous application in the hair field, in particular for the form retention of the hairstyle or the shaping of the hair, or also the care, cosmetic treatment or cleaning of the hair. The hair compositions are preferably shampoos, hair conditioners, styling or care gels, care lotions or creams, conditioners, hair setting lotions, blow drying lotions, fixing and styling compositions, such as lacquers or sprays, hair restructuring lotions, lotions or gels for combating hair loss, antiparasitic shampoos, antidandruff lotions or shampoos or shampoos for treating seborrhoea. The lotions can be packaged in various forms, in particular in vaporizers, pump-action sprays or aerosol containers, in order to provide for application of the composition in the vaporized form or in the foam form.

It can also be provided in the form of a hair dyeing product, in particular oxidation dyeing or direct dyeing product, optionally in the form of a colouring shampoo, in the form of a perming, hair straightening or bleaching composition, or also in the form of a rinse-out composition, to be applied before or after a dyeing, bleaching, perming or hair straightening operation or also between the two stages of a perming operation or hair straightening operation.

The composition according to the invention can also be provided in the form of a care composition, in particular a moisturizing composition, for the skin of the body or of the face, the lips and/or the superficial body growths, in particular of a care product intended to cosmetically treat the skin and in particular to moisturize it, to smooth it, to depigment it, to nourish it, to protect it from sunlight or to confer a specific cosmetic treatment on it. Thus, it can be a lip care base, a fixing base for lipsticks, an antisun protection or artificial tanning composition, a care (day, night, antiageing or moisturizing) composition for the face, a matifying composition, a composition for cleaning the skin, for example a make-up-removing product or a bath or shower gel or a cleaning bar or soap, a body hygiene composition, in particular a deodorant or antiperspirant product, or also a depilatory composition, an aftershave gel or an aftershave lotion.

It can also be provided in the form of a product for making up the skin of the body or of the face, the lips, the eyelashes, the nails or the hair, in particular a foundation, a blusher, a face powder, an eyeshadow, a concealer, an eyeliner, a mascara, a lipstick, a lip gloss, a lip pencil, a nail varnish, a nail care product or a product for the temporary tattooing of the skin of the body.

More particularly still, the composition according to the invention has an advantageous application in the care and the cosmetic treatment, in particular the protection, of the hair, especially weakened and/or damaged hair, for example hair weakened and/or damaged by chemical or mechanical treatments; use may in particular be made of the compounds according to the invention in post-treatment, after a stage of dyeing, bleaching or straightening the hair.

A subject-matter of the invention is thus a method for the cosmetic treatment, in particular for the making up, the care, the cleaning, the dyeing or the shaping, of keratinous substances, in particular of the skin of the body or of the face, the lips, the nails, the hair and/or the eyelashes, comprising the application, to the said substances, of a cosmetic composition comprising at least one compound according to the invention.

Preferably, it is a cosmetic treatment method for the conditioning of the hair, in particular for providing it with suppleness, disentangling, smoothing and/or the ability to be combed or for improving the suppleness, the disentangling, the smoothing and/or the ability to be combed thereof.

The application of the composition can optionally be followed by a rinsing stage and/or optionally by a heat treatment stage.

The invention is illustrated in more detail in the following examples.

General Synthesis Method for the Compounds in which R4 Comprises a Ring in the Chain (Examples 1 to 4)

The synthesis of the carboxylic acids comprising one or more cyclopropane rings in the chain is carried out according to the procedures described in the papers: Takai et al., JOC, 1994, 59, 2671-2673; and Tanaka et al., Bioorganic Chemical Chemistry Letters, 13, 2003, 1037-1040.

The synthesis scheme can be illustrated as follows:

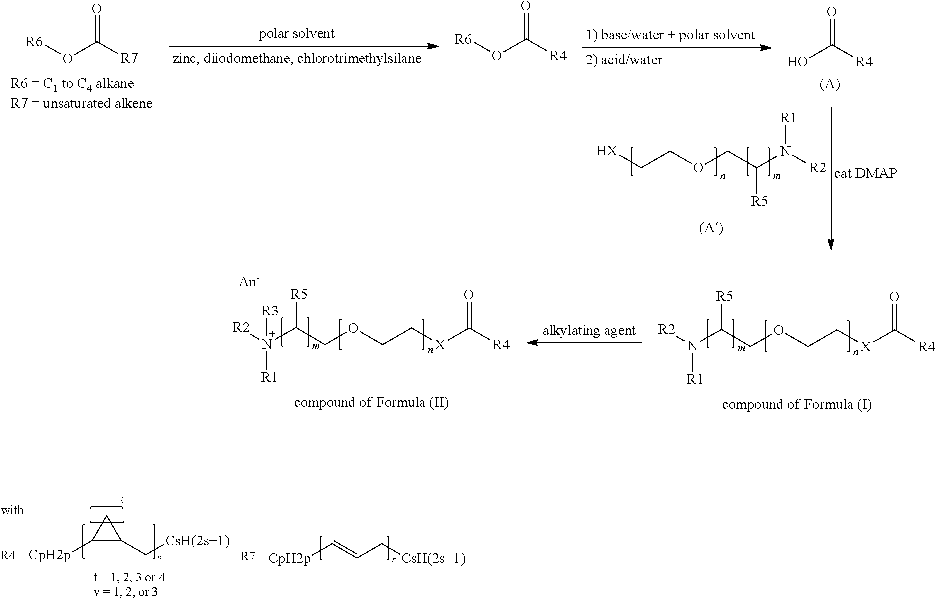

Generally, the carboxylic acid A, the amino alcohol A′ and a catalyst (for example N,N-dimethyl-aminopyridine) are stirred, under an inert atmosphere, in an organic solvent, for example anhydrous dichloromethane, at a temperature of between −5° C. and 10° C. A coupling agent, for example 1-(3-dimethylamino-propyl)-3-ethylcarbodiimide hydrochloride, can be slowly added. The reaction medium is preferably stirred at this temperature for one hour before being brought back to ambient temperature. On completion of the reaction, the reaction medium is hydrolyzed and then, after extraction by an organic solvent, for example dichloromethane, the organic phase is washed using an aqueous solution, dried over a metal sulphate (for example sodium sulphate or magnesium sulphate) and concentrated under reduced pressure. The compound obtained is of formula (I); it can be dried under vacuum in the presence of P₂O₅.

The quaternized compound of formula (II) can be obtained by reacting the compound (I) and an alkylating agent, such as, for example, methyl iodide, dimethyl sulphate, ethyl iodide, diethyl sulphate or a haloalkane. If necessary, an ion exchange can be carried out on conclusion of the reaction, by contact with an ion-exchange resin chosen according to the exchanges desired. These resins are, for example, IRA 402 (exchange of alkyl sulphates to give chlorides) or IRA 400 (exchange of iodide to give chloride).

General Synthesis Method for the Compounds in which R4 Comprises a Ring at the Chain End (Examples 5 to 12), in Particular a Ring Derived from Chaulmoogra Oil

The synthesis scheme is as follows:

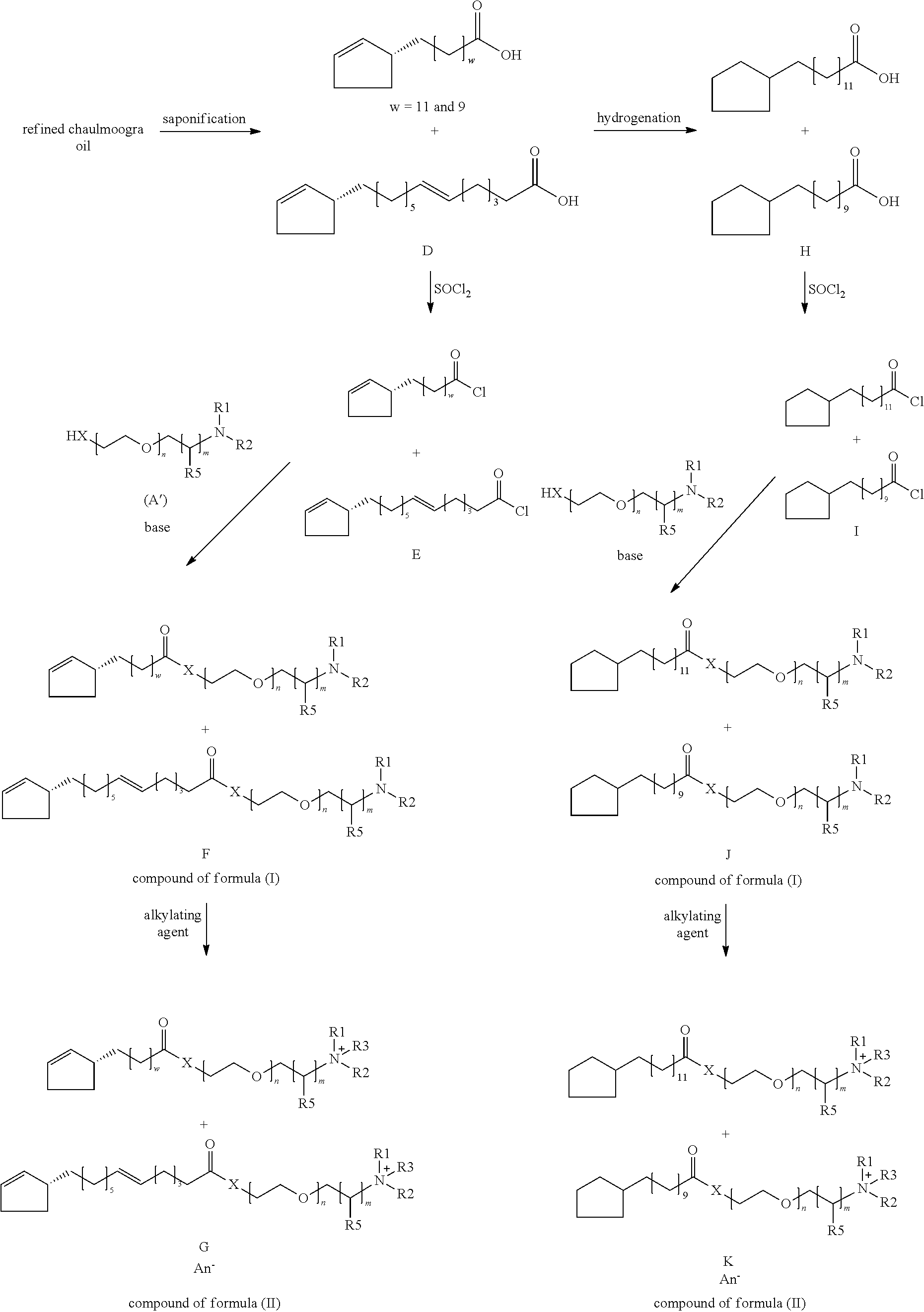

The mixture of acids D resulting from the chaulmoogra oil is obtained by saponification of the said oil.

After activation of the carboxyl functional group by methods known to a person skilled in the art, for example acid chloride of formula E, these acids are condensed with an alcohol, a thiol or a primary amine A′ to result in esters, thioesters or amides of formula (I). These compounds can subsequently be reacted with alkylating agents, such as those described above, to result in the quaternized compounds of formula (II).

The corresponding saturated compounds can be prepared according to the same method from the mixture of hydrogenated acids H, which essentially comprises only 11-cyclopentylundecanoic acid and 13-cyclopentyl-tridecanoic acid.

### EXAMPLES 1 AND 2

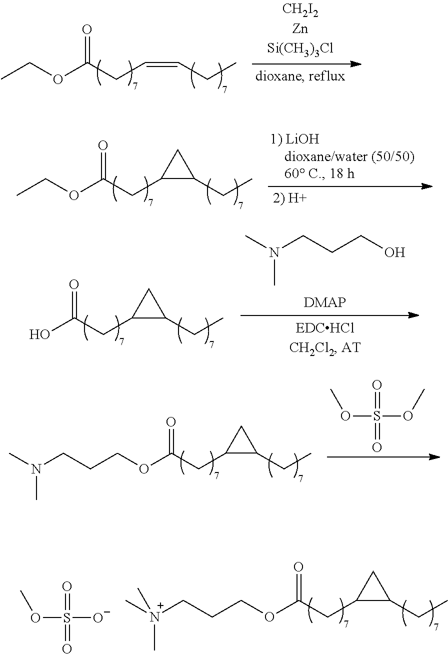

### Example 1

### Synthesis of 3-(dimethylamino)propyl 8-(2-octylcyclopropyl)octanoate

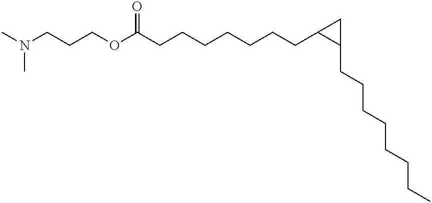

3.6 g of 8-(2-octylcyclopropyl)octanoic acid (12.14 mmol), 1.447 ml of 3-(N,N-dimethylamino)-1-propanol (12.14 mmol) and then 100 ml of dichloromethane were introduced into a 250 ml reactor equipped with a reflux condenser, a thermometer, a dropping funnel and an argon inlet. After having cooled to a temperature of approximately 5° C., 0.297 g of 4-dimethylaminopyridine (2.43 mmol) were introduced and then, after stirring for 30 minutes, 4.655 g of 1-ethyl-3-(3-dimethylaminopropyl)carbodiimide hydrochloride (24.28 mmol) were added in small amounts so as to keep the temperature below 10° C.

On completion of the addition, the reaction medium was allowed to return to ambient temperature and was then maintained at ambient temperature for 5 hours. The reaction mixture was subsequently poured onto 300 ml of water and then extracted with 3 times 100 ml of dichloromethane. After washing the organic phase with 100 ml of a saturated ammonium chloride solution, the organic phase was dried over sodium sulphate, filtered on a sintered glass funnel and then evaporated under reduced pressure to produce 4.45 grams of the expected product in a form of a yellow oil with a yield of 96%.

¹H NMR spectrum, 400 MHz, and ¹³C NMR spectrum, 100 MHz:

in accordance
Mass spectrum: (m/z) [M+H]⁺=382

### Example 2

### Synthesis of N,N,N-trimethyl-3-{[8-(2-octyl-cyclopropyl)]octanoyl]oxy}propan-1-aminium methyl sulphate

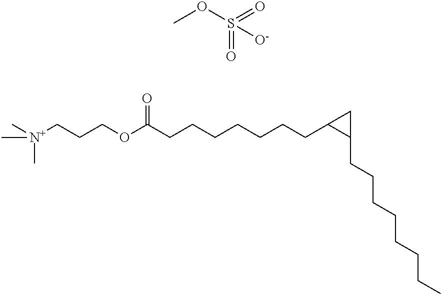

2.2 grams of 3-(dimethylamino)propyl 8-(2-octyl-cyclopropyl)octanoate prepared in Example 1 (5.76 mmol) and then 15 ml of tetrahydrofuran, 1.222 grams of sodium carbonate (11.53 mmol) and 0.518 ml of dimethyl sulphate (5.48 mmol) were introduced into a 50 ml reactor equipped with a reflux condenser, a thermometer and an argon inlet. The reaction mixture was subsequently kept stirred at ambient temperature for 24 hours and then filtered on a sintered glass funnel. The filtrate was subsequently poured onto 100 ml of isopropyl ether. This washing operation was repeated 3 times and then the precipitate formed was filtered off on a sintered glass funnel to produce, after drying, 2.77 grams of an orange wax with a yield of 95%.

¹H NMR spectrum, 400 MHz, and ¹³C NMR spectrum, 100 MHz: in accordance

Mass spectrum: (m/z) [M]⁺=396

### Example 3

### Synthesis of 2-[2-(diethylamino)ethoxy]ethyl 8-(2-octylcyclopropyl)octanoate

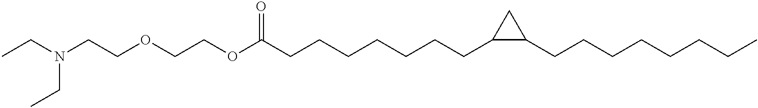

### Stage 1: Synthesis of the acid chloride of 8-(2-octyl-cyclopropyl)octanoic acid

16.3 g of 8-(2-octylcyclopropyl)octanoic acid, then 100 ml of toluene and then 10.45 ml of oxalyl chloride were introduced into a 500 ml reactor equipped with a reflux condenser, a thermometer and an argon inlet. The reaction mixture was kept stirred at ambient temperature for 2 hours. The progress of reaction was monitored by NMR after withdrawing an aliquot and evaporating under reduced pressure. After evaporating the reaction medium under reduced pressure, 17.2 grams of an orangey yellow oil were obtained, corresponding to the acid chloride of 8-(2-octylcyclopropyl)octanoic acid, with a quantitative yield.

### Stage 2

2.63 g of predried sodium carbonate (24.81 mmol), 100 ml of methyltetrahydrofuran and 2.13 ml of 2-(2-diethylaminoethoxy)ethanol (12.4 mmol) were introduced into a 500 ml reactor equipped with a reflux condenser, a thermometer and an argon inlet. The reaction medium was subsequently cooled to a temperature of approximately 5° C. and then a solution of 4.69 grams of the chloride of 8-(2-octylcyclopropyl)octanoic acid prepared beforehand (14.88 mmol), dissolved in 30 ml of methyltetrahydrofuran, was added dropwise, so as to maintain a temperature of approximately 5° C. On completion of the addition, the reaction medium was subsequently allowed to return to ambient temperature and then kept stirred for 12 hours. The reaction medium was subsequently filtered through a filter paper and then the organic phase was washed with water. The organic phase was subsequently washed with 100 ml of a saturated sodium hydrogencarbonate solution and dried over sodium sulphate and then, after filtration, the residue was formed into a paste with 10 grams of basic alumina. Filtration was subsequently carried out, followed by evaporation under reduced pressure, to produce 5.68 g of a yellow oil.

This oil was subsequently purified on a silica column in a dichloromethane/methanol mixture to produce 3.11 g of 2-[2-(diethylamino)ethoxy]ethyl 8-(2-octyl-cyclopropyl)octanoate in the form of a translucent oil with a yield of 60%.

¹H NMR spectrum, 400 MHz, and ¹³C NMR spectrum, 100 MHz: in accordance

Mass spectrum: (m/z) [M+H]⁺=441

### Example 4

### Synthesis of N,N,N-triethyl-2-(2-{[8-(2-octylcyclopropyl)octanoyl]oxy}ethoxy)ethanaminium ethyl sulphate

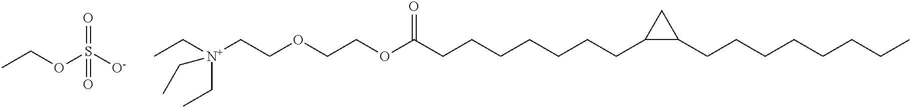

2 grams of 2-[2-(diethylamino)ethoxy]ethyl 8-(2-octylcyclopropyl)octanoate prepared in Example 3 (4.55 mmol) and 1.19 ml of diethyl sulphate (9.1 mmol) were introduced into a 50 ml reactor equipped with a reflux condenser, a thermometer and an argon inlet. The reaction mixture was subsequently maintained at ambient temperature with stirring for 24 hours. 10 ml of dichloromethane were subsequently added and then purification was carried out on a silica column in a dichloromethane/methanol/aqueous ammonia mixture.

After evaporating the pure fractions, 2 g of N,N,N-triethyl-2-(2-{[8-(2-octylcyclopropyl)octanoyl]-oxy}ethoxy)ethanaminium ethyl sulphate were obtained in the form of a coloured paste with a yield of 74%.

¹H NMR spectrum, 400 MHz, and ¹³C NMR spectrum, 100 MHz: in accordance

Mass spectrum: (m/z) [M]⁺=468

### Example 5

### Stage 1

### Synthesis of the Mixture of Acids Resulting From Chaulmoogra Oil (Mixture Essentially Composed of Hydnocarpic Acid, Gorlic Acid and Chaulmoogric Acid)

600 ml of 95% ethanol comprising 150 g of potassium hydroxide were added to a suspension of 300 g of refined chaulmoogra oil (ID Bio) in 900 ml of water. The reaction mixture was subsequently brought with stirring to a temperature of 80° C. for 5 hours and were then left overnight at ambient temperature. 500 ml of ethanol/water mixture were subsequently evaporated off under reduced pressure, 1 litre of water was then added and extraction was carried out with 3 times 300 ml of ethyl ether in order to remove the non-saponifiable impurities. The reaction medium was cooled to 5° C. with an ice bath and then a 37% hydrochloric acid solution was added dropwise until a final pH of 1.35 was obtained. The precipitate obtained was filtered off on a sintered glass funnel and then washed with 5 litres of water in order to obtain aqueous wash liquors at neutral pH. The precipitate was subsequently dried in an oven at 35° C. to produce 290 g of expected mixture of acids (D).

¹H NMR spectrum, 400 MHz, and ¹³C NMR spectrum, 100 MHz: in accordance

GC: determination of the percentage of fatty acid:
hydnocarpic acid: 46.6%
gorlic acid: 10.3%
chaulmoogric acid: 29.3%

### Stage 2

### Synthesis of the Chaulmoogric Acid Chloride+Hydnocarpic Acid Chloride+Gorlic Acid Chloride Mixture

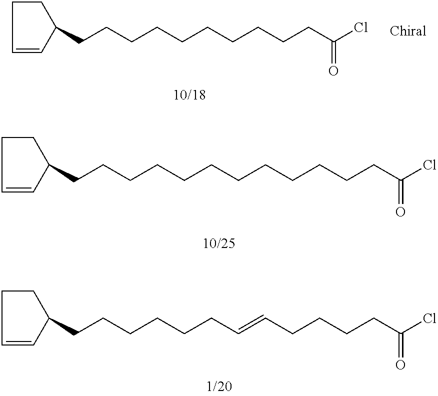

6 g of the mixture of acids obtained in Stage 1 were introduced into a 250 ml reactor equipped with a reflux condenser, a thermometer and an argon inlet and then 100 ml of dichloromethane were added. 2.96 ml of oxalyl chloride were subsequently added and then the reaction mixture was brought to reflux for 4 hours. Evaporation was subsequently carried out under reduced pressure to produce 6.4 grams of an oil slightly orange in colour with a yield of the order of 100%. The mixture of acid chlorides is subsequently reacted as is.

¹H NMR spectrum, 400 MHz, and ¹³C NMR spectrum: 100 MHz: in accordance

### Stage 3: Synthesis of the 3-(dimethylamino)propyl 13-[(1R)-cyclopent-2-en-1-yl]tridecanoate+3-(dimethyl-amino)propyl 11-[(1R)-cyclopent-2-en-1-yl]undecanoate+3-(dimethylamino)propyl (6E)-13-[(1R)-cyclopent-2-en-1-yl]tridec-6-enoate mixture

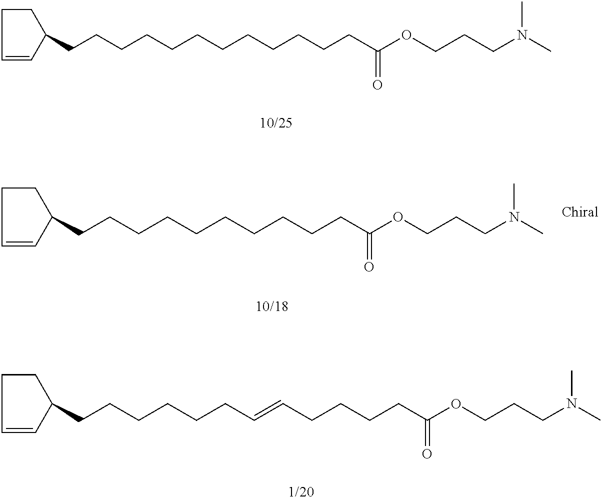

1.17 g of 3-dimethylaminopropanol (0.01126 mol), 1.486 g of N,N-diisopropylethylamine (0.01126 mol) and then 100 ml of dichloromethane were introduced into a 250 ml reactor equipped with a reflux condenser, a thermometer, a dropping funnel and an argon inlet. After having cooled to a temperature of approximately 5° C., a solution of 3 g of acid chlorides prepared in Stage 2 (0.01126 mol) dissolved in 50 ml of dichloromethane was introduced dropwise so as to maintain a temperature of less than 10° C.

On completion of the addition, the reaction medium was allowed to return to ambient temperature and was then kept at ambient temperature for 3 hours. The reaction mixture was subsequently poured onto 300 ml of slightly acidic water (pH 5) and then extracted with 3 times 100 ml of dichloromethane. After washing the organic phase with 100 ml of a saturated ammonium chloride solution, the organic phase was dried over sodium sulphate, filtered on a sintered glass funnel and then evaporated under reduced pressure. The crude product obtained was subsequently purified on a basic alumina column and then, after evaporating, 2.5 grams of the expected product were obtained in the form of a white powder with a yield of 63%.

¹H NMR spectrum, 400 MHz, and ¹³C NMR spectrum, 100 MHz: in accordance

### Example 6

### Synthesis of the 3-({13-[(1R)-cyclopent-2-en-1-yl]tridecanoyl}oxy)-N,N,N-trimethylpropan-1-aminium methyl sulphate+3-({11-[(1R)-cyclopent-2-en-1-yl]undecanoyl}oxy)-N,N,N-trimethylpropan-1-aminium methyl sulphate+3-({(6E)-13-[(1R)-cyclopent-2-en-1-yl]tridec-6-enoyl}oxy)-N,N,N-trimethylpropan-1-aminium methyl sulphate mixture

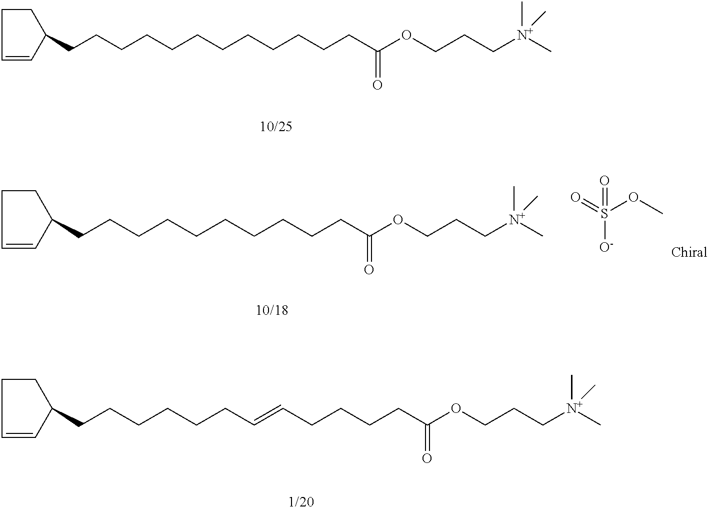

3.1 g of the derivative obtained in Stage 3 of Example 5 (0.0088 mol) were introduced into a 250 ml reactor equipped with a reflux condenser, a thermometer and an argon inlet and then 50 ml of dichloromethane were added. 1.11 grams of dimethyl sulphate (0.0088 mol) were subsequently added and then the reaction mixture was brought with stirring to ambient temperature for 12 hours. It was subsequently evaporated under reduced pressure and then the crude product obtained was washed with isopropyl ether to produce 3.43 grams of the expected product in the form of a beige paste with a yield of 81%.

¹H NMR spectrum, 400 MHz, and ¹³C NMR spectrum, 100 MHz: in accordance

Mass spectrum: (m/z): [M]⁺=352 and 380

### Example 7

### Synthesis of the 11-cyclopentyl-N-[3-(dimethylamino)propyl]undecanamide+13-cyclopentyl-N-[3-(dimethylamino)propyl]tridecanamide mixture

### Stage 1: Synthesis of the mixture of 11-cyclopentyl-undecanoic acid and 13-cyclopentyltridecanoic acid

50 grams of the mixture of acids obtained in Stage 1 of Example 5, 6 grams of 50% wet 5% palladium-on-charcoal and 300 ml of ethanol were introduced into a hydrogenator. After degassing with argon, hydrogen was introduced at a flow rate of 5 ml/min, a pressure of 6 bar and a temperature of 40° C. After reacting for 1 h 30, no more hydrogen consumption is observed. Stirring was halted, purging was then carried out with argon and then, after filtering through celite, the alcohol phase was evaporated under reduced pressure to produce 46 grams of a waxy white powder with a yield of the order of 95%.

¹H NMR spectrum, 400 MHz, and ¹³C NMR spectrum, 100 MHz: in accordance

### Stage 2: Synthesis of the mixture of 11-cyclopentyl-undecanoic acid chloride and 13-cyclopentyltridecanoic acid chloride (50/50)

3.3 g of the mixture of acids prepared in Stage 1 (mean MW=270, i.e. 0.0185 mol) were introduced into 100 ml of dichloromethane in a 250 ml reactor equipped with a reflux condenser, an acid vapour trapping system (sodium hydroxide sol), a thermometer and an argon inlet. 5 ml of thionyl chloride (0.0685 mol) were subsequently added and the reaction mixture was brought to reflux for 4 hours. After one night at ambient temperature, the reaction medium was evaporated to produce 3.5 grams of a light yellow oil with a yield of the order of 100% (mean MW=286.9).

### Stage 3: Synthesis of the 11-cyclopentyl-N-[3-(dimethylamino)propyl]undecanamide+13-cyclopentyl-N-[3-(dimethylamino)propyl]tridecanamide mixture

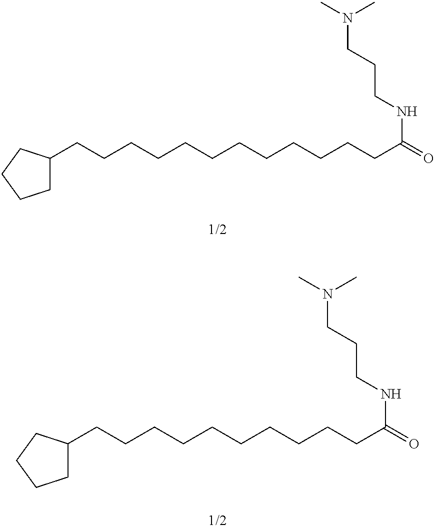

1.28 g of N,N-dimethylaminopropylamine (0.0125 mol), 1.62 g of N,N-diisopropylethylamine (0.0125 mol) and then 100 ml of dichloromethane were introduced into a 250 ml reactor equipped with a reflux condenser, a thermometer, a dropping funnel and an argon inlet. After having cooled to a temperature of approximately 5° C., a solution of 3.5 g of acid chlorides prepared in Stage 2 (0.0122 mol) dissolved in 50 ml of dichloromethane was introduced dropwise so as to keep the temperature below 10° C.

On completion of the addition, the reaction medium was allowed to return to ambient temperature and was then kept at ambient temperature for 3 hours. The reaction mixture was subsequently poured onto 300 ml of slightly acidic water (pH 5) and then extracted with 3 times 100 ml of dichloromethane. After washing the organic phase with 100 ml of a saturated ammonium chloride solution, the organic phase was dried over sodium sulphate, filtered on a sintered glass funnel and then evaporated under reduced pressure to produce 3.7 grams of the expected product in the form of a white powder with a yield of 85%.

¹H NMR spectrum, 400 MHz, and ¹³C NMR spectrum, 100 MHz: in accordance

Mass spectrum: (m/z) [M+H]⁺=339 and 367

### Example 8

### Synthesis of the 3-[(11-cyclopentylundecanoyl)amino]-N,N,N-trimethylpropan-1-aminium methyl sulphate+3-[(13-cyclopentyl-tridecanoyl)amino]-N,N,N-trimethylpropan-1-aminium methyl sulphate mixture

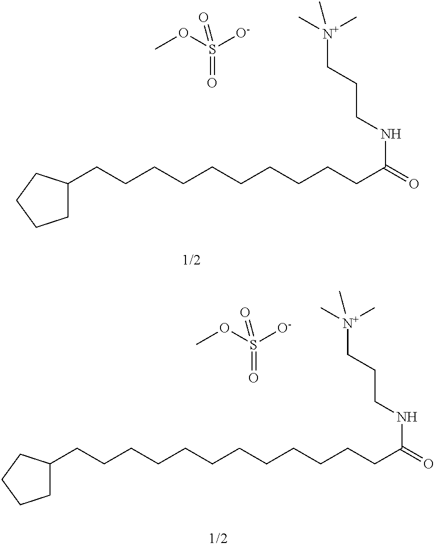

3.1 g of the mixture prepared in Stage 3 of Example 7 (0.0088 mol) and then 2 ml of dimethyl sulphate (0.0211 mol) were introduced into a 50 ml reactor equipped with a reflux condenser, a thermometer and an argon inlet. The reaction mixture was subsequently maintained at ambient temperature with stirring for 24 hours and then poured onto 100 ml of isopropyl ether. This washing operation was repeated 3 times and then the precipitate formed was filtered off on a sintered glass funnel to produce, after drying, 4 g of a brownish paste.

¹H NMR spectrum, 400 MHz, and ¹³C NMR spectrum, 100 MHz: in accordance but a trace of the compound of Example 7 remains.

Mass spectrum: (m/z) [M]⁺=353 and 381

### Example 9

### Synthesis of the 2-[2-(diethylamino)ethoxy]ethyl 11-cyclopentylundecanoate+2-[2-(diethylamino)ethoxy]ethyl 13-cyclopentyl-tridecanoate mixture

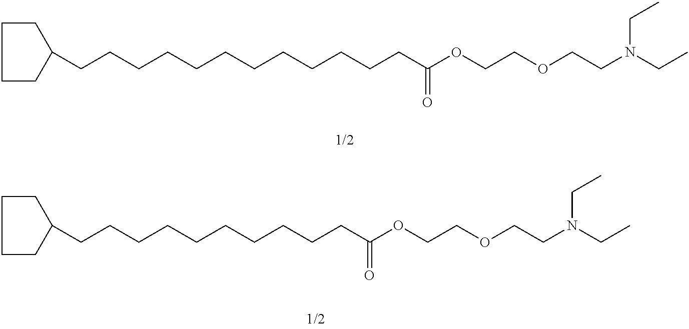

2.13 g of 2-(2-diethylaminoethoxy)ethanol (0.0132 mol), 1.4 g of triethylamine (0.0138 mol) and then 100 ml of dichloromethane were introduced into a 250 ml reactor equipped with a reflux condenser, a thermometer, a dropping funnel and an argon inlet. After having cooled to a temperature of approximately 5° C., a solution of 3.6 g of acid chlorides prepared in Stage 2 of Example 7 (0.0126 mol) dissolved in 50 ml of dichloromethane was introduced dropwise so as to keep the temperature below 10° C.

On completion of the addition, the reaction medium was allowed to return to ambient temperature and then kept at ambient temperature for 3 hours. The reaction mixture was subsequently poured onto 300 ml of slightly acidic water (pH 5) and then extracted with 3 times 100 ml of dichloromethane. After washing the organic phase with 100 ml of a saturated ammonium chloride solution, the organic phase was dried over sodium sulphate, filtered on a sintered glass funnel and then evaporated under reduced pressure to produce 4.26 g of the expected product in the form of a brown paste with a yield of 82%.

¹H NMR spectrum, 400 MHz, and ¹³C NMR spectrum: 100 MHz: in accordance

### Example 10

### Synthesis of the 2-{2-[(11-cyclopentylundecanoyl)oxy]ethoxy}-N,N,N-triethyl-ethanaminium ethyl sulphate+2-{2-[(13-cyclopentyl-tridecanoyl)oxy]ethoxy}-N,N,N-triethylethanaminium ethyl sulphate mixture

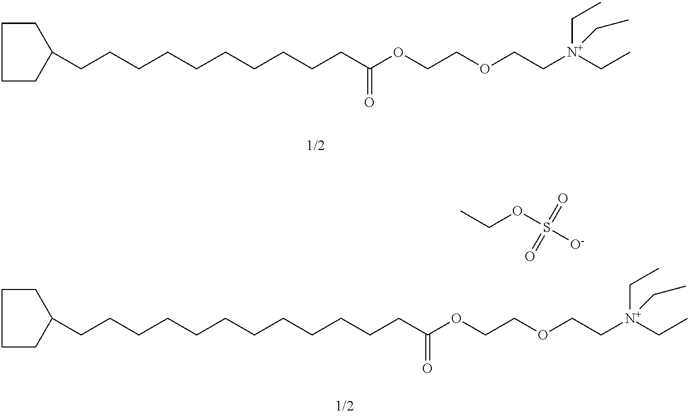

3.8 g of the mixture obtained in Example 9 (0.00925 mol) and then 5 ml of diethyl sulphate (0.037 mol) were introduced into a 50 ml reactor equipped with a reflux condenser, a thermometer and an argon inlet. The reaction mixture was subsequently maintained at ambient temperature with stirring for 24 hours and then poured onto 100 ml of isopropyl ether. This operation of washing with isopropyl ether was repeated 3 times and then the precipitate formed was filtered off on a sintered glass funnel to produce, after drying, 4 g of a brownish paste with a yield of 98%.

¹H NMR spectrum, 400 MHz, and ¹³C NMR spectrum, 100 MHz: in accordance

Mass spectrum: (m/z): [M]⁺=426 and 454

### Example 11

### Synthesis of the 13-[(1R)-cyclopent-2-en-1-yl]-N-[3-(dimethylamino)propyl]tridecanamide+11-[(1R)-cyclopent-2-en-1-yl]-N-[3-(dimethylamino)propyl]-undecanamide+(6E)-13-[(1R)-cyclopent-2-en-1-yl]-N-[3-(dimethylamino)propyl]tridec-6-enamide mixture

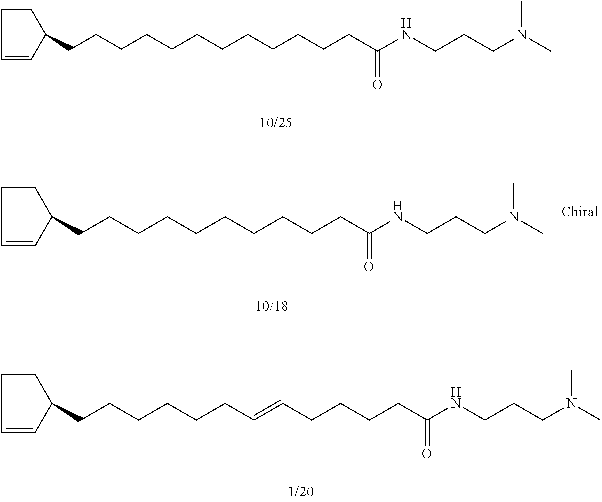

2.28 g of 3-dimethylaminopropylamine (0.0223 mol), 2.88 g of N,N-diisopropylethylamine (0.0223 mol) and then 100 ml of dichloromethane were introduced into a 250 ml reactor equipped with a reflux condenser, a thermometer, a dropping funnel and an argon inlet. After having cooled to a temperature of approximately 5° C., a solution of 6.35 g of acid chlorides prepared in Stage 2 of Example 5 (0.00223 mol) dissolved in 50 ml of dichloromethane was introduced dropwise so as to keep the temperature below 10° C.

On completion of the addition, the reaction medium was allowed to return to ambient temperature and was then kept at ambient temperature for 4 hours. The reaction mixture was subsequently poured onto 100 ml of slightly acidic water (pH 5) and then extracted with 3 times 100 ml of dichloromethane. After washing the organic phase with 100 ml of a saturated ammonium chloride solution, the organic phase was dried over sodium sulphate, filtered on a sintered glass funnel and then evaporated under reduced pressure. The crude product obtained was subsequently purified on a basic alumina column and then, after evaporation, 5.55 g of the expected product were obtained in the form of a beige wax with a yield of 70.8%.

¹H NMR spectrum, 400 MHz, and ¹³C NMR spectrum, 100 MHz: in accordance

Mass spectrum: (m/z): [M+H]⁺=337 and 365

### Example 12

### Synthesis of the 3-({13-[(1R)-cyclopent-2-en-1-yl]tridecanoyl}amino)-N,N,N-trimethylpropan-1-aminium methyl sulphate+3-({11-[(1R)-cyclopent-2-en-1-yl]undecanoyl}amino)-N,N,N-trimethylpropan-1-aminium methyl sulphate+3-({(6E)-13-[(1R)-cyclopent-2-en-1-yl]tridec-6-enoyl}amino)-N,N,N-trimethylpropan-1-aminium methyl sulphate mixture

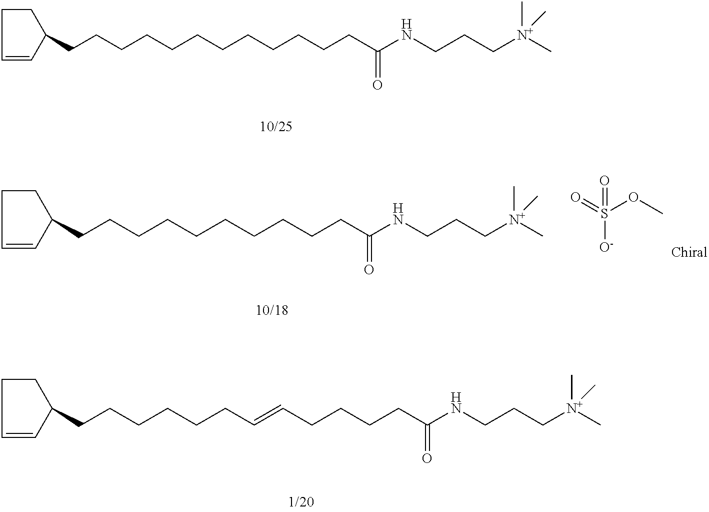

2.51 g of the derivative obtained in Example 11 (0.00715 mol) were introduced into a 250 ml reactor equipped with a reflux condenser, a thermometer and an argon inlet and then 50 ml of dichloromethane were added. 1.8 g of dimethyl sulphate (0.0143 mol) were subsequently added and then the reaction mixture was brought with stirring to ambient temperature for 12 hours. It was subsequently evaporated under reduced pressure and then the crude product obtained was washed with isopropyl ether to produce 3.3 g of the expected product in the form of a beige solid with a yield of 96%.

¹H NMR spectrum, 400 MHz, and ¹³C NMR spectrum, 100 MHz: in accordance

Mass spectrum: (m/z): [M]⁺=351 and 379

### Example 13

The following hair cosmetic composition is prepared (% by weight):

3% of N,N,N-trimethyl-3-{[8-(2-octylcyclopropyl)-octanoyl]oxy}propan-1-aminium methyl sulphate (Example 2)

q.s. for 100% of water.

A lock of bleached hair is treated by immersion in the hair cosmetic composition in a proportion of 15 g of solution for 0.5 g of hair. The treatment is carried out at 30° C. for 15 minutes and it is followed by a rinsing with water. It is observed that the wet lock has a smooth feel, is supple and is easy to disentangle.

1. A compound represented by formula (I) or (II): 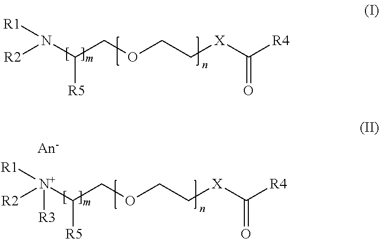
in which:
m is an integer between 1 and 10;
n is an integer between 0 and 10;
X represents O, NH or S;
R1, R2 and R3 denote, independently of one another, a linear C₁-C₂₂ or branched C₃-C₂₈ alkyl group or a linear C₂-C₂₂ or branched C₃-C₂₈ alkenyl group, these groups being optionally substituted by one or more identical or different radicals chosen from hydroxyl (—OH) and amino (—NRR′) radicals, with R and R′ chosen, independently of one another, from H and C₁-C₆ alkyl; or else
R1 and R2 form, with the nitrogen atom to which they are connected, a saturated or unsaturated carbon-comprising heterocycle comprising 5 or 6 ring members, wherein one or two nonadjacent carbon atoms may optionally replaced by an oxygen, nitrogen (—NR″), with R″=H or C₁-C₂₂ alkyl, or sulphur atom; wherein said heterocycle may optionally to be substituted by one or more identical or different radicals chosen from aryl, C₁-C₂₂ alkyl, hydroxyl or amino (—NRR′) radicals, with R and R′ chosen, independently of one another, from H and C₁-C₆ alkyl; and R3 has the above definition, if appropriate;
R4 denotes:
either a linear or branched C₄-C₂₂ alkyl radical (saturated) comprising, as interruption in the chain, from one to three identical or different and saturated or unsaturated carbon-comprising rings each comprising 3, 4, 5 or 6 ring members;
the said ring(s) being optionally substituted by an aryl, itself optionally substituted by one or more identical or different radicals chosen from C₁-C₆ alkyl, hydroxyl (—OH) and amino (—NRR′) radicals, with R and R′ chosen, independently of one another, from H and C₁-C₆ alkyl;
it being understood that, when R4 denotes an alkyl radical including 2 or 3 carbon-comprising rings, the said rings are separated from one another by a divalent methylene or ethylene radical;
or a linear or branched C₄-C₂₂ alkyl or alkenyl radical (saturated or unsaturated) comprising, at the chain end, a saturated or unsaturated and non-aromatic carbon-comprising ring comprising 5 or 6 ring members;
R5 denotes a hydrogen atom or an OR or NRR′ radical with R and R′ chosen, independently of one another, from H and C₁-C₆ alkyl; it being understood that, when m is greater than or equal to 2, the R5 radicals are identical or different; and
An⁻ denotes an organic or inorganic anion or a mixture of organic or inorganic anions, in order to ensure the electrical neutrality of the compounds of formula (II).

2. The compound according to claim 1, in which X represents O or NH.

3. The compound according to claim 1, in which R1 denotes a linear C₁-C₄ alkyl or branched C₃-C₄ alkyl group or a linear C₂-C₄ alkenyl or branched C₃-C₄ alkenyl group.

4. The compound according to claim 1, in which R2 denotes a linear C₁-C₄ alkyl or branched C₃-C₄ alkyl group or a linear C₂-C₄ alkenyl or branched C₃-C₄ alkenyl group.

5. The compound according to claim 1, in which R3 denotes a linear C₁-C₂₂ alkyl or branched C₃-C₂₂ alkyl group or a linear C₂-C₂₂ alkenyl or branched C₃-C₂₂ alkenyl group.

6. The compound according to claim 1, in which R4 denotes:
either a linear C₄-C₂₂ alkyl radical comprising, as interruption in the chain, from one to three identical or different and saturated or unsaturated carbon-comprising rings each comprising 3, 4, 5 or 6 ring members; preferably a linear C₁₂-C₂₁ alkyl radical comprising, as interruption in the chain, from one to three identical and saturated carbon-comprising rings each comprising 3, 4, 5 or 6 ring members; the said alkyl radical very particularly comprising, as interruption, from one to three cyclopropyl rings, it being understood that, when 2 or 3 rings are present, they are separated from one another by a divalent methylene —CH₂— radical;
or a linear or branched C₄-C₂₁ alkyl or alkenyl radical (saturated or unsaturated) comprising, at the chain end, a saturated or unsaturated and non-aromatic carbon-comprising ring comprising 5 ring members.

7. The compound according to claim 1, chosen from the following compounds and their salts or solvates, alone or as a mixture:

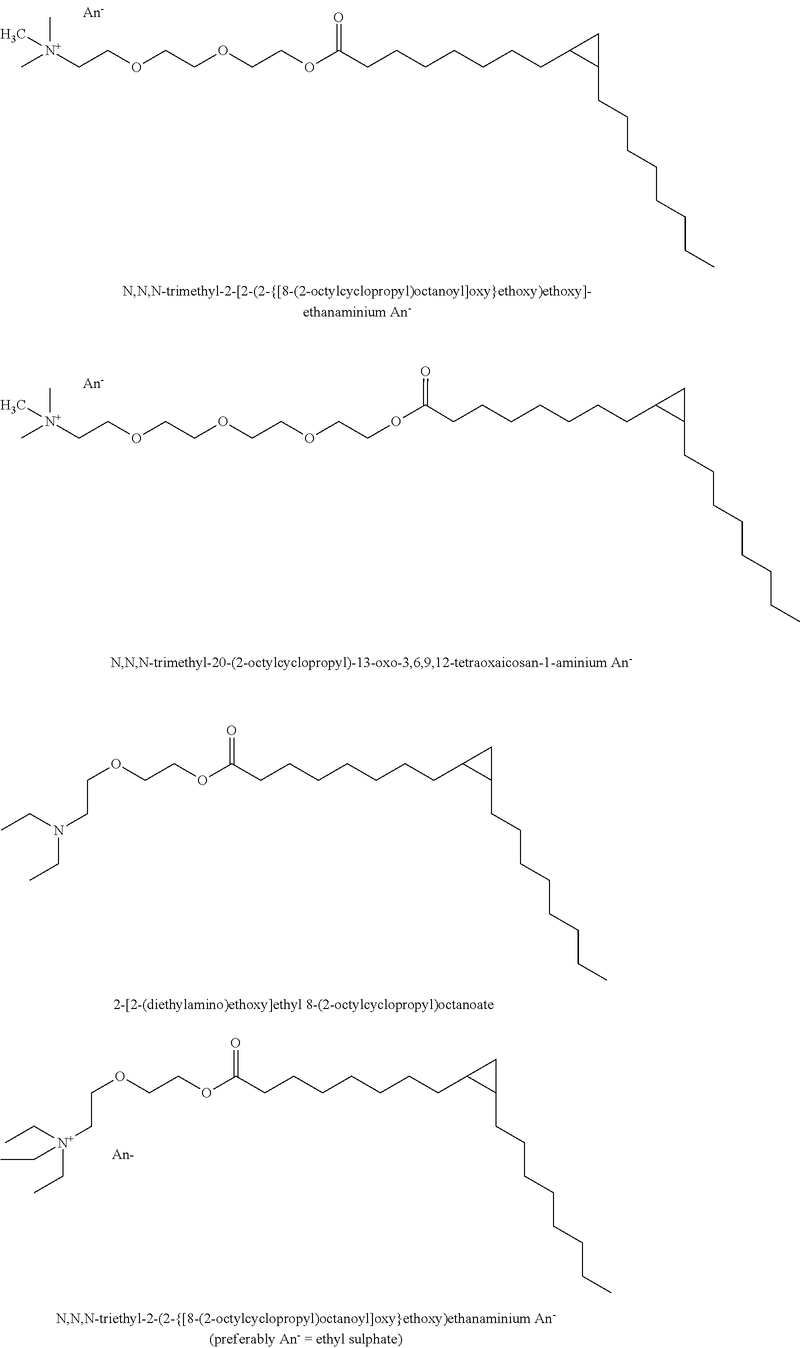

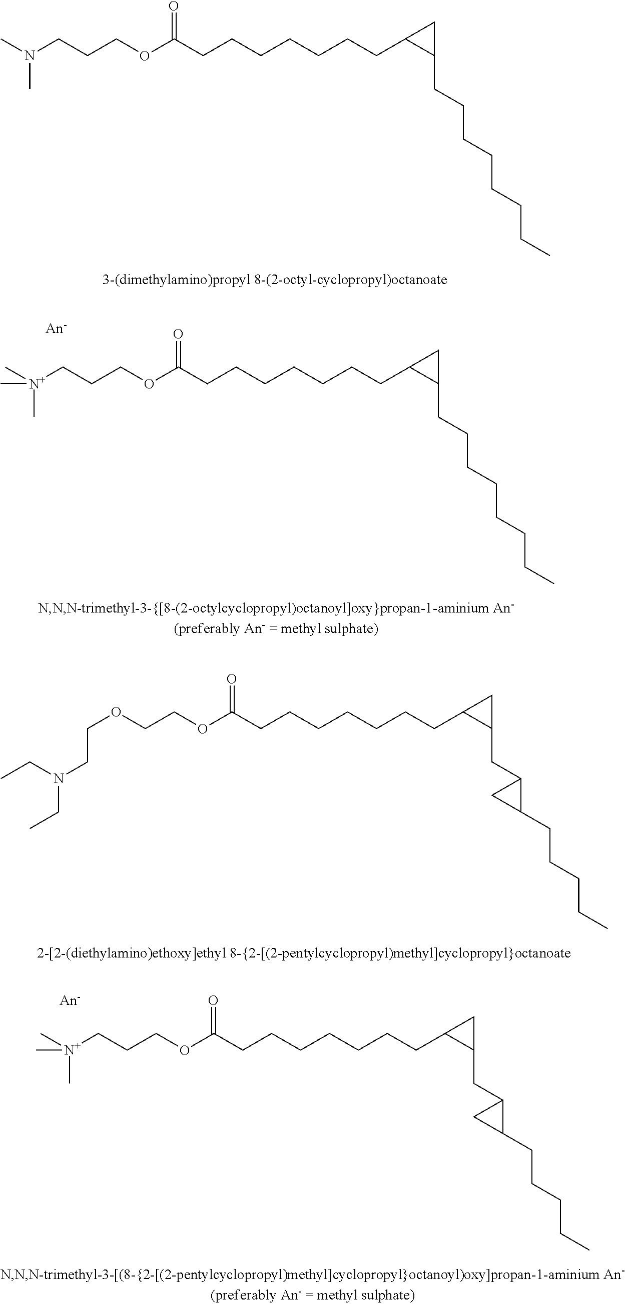

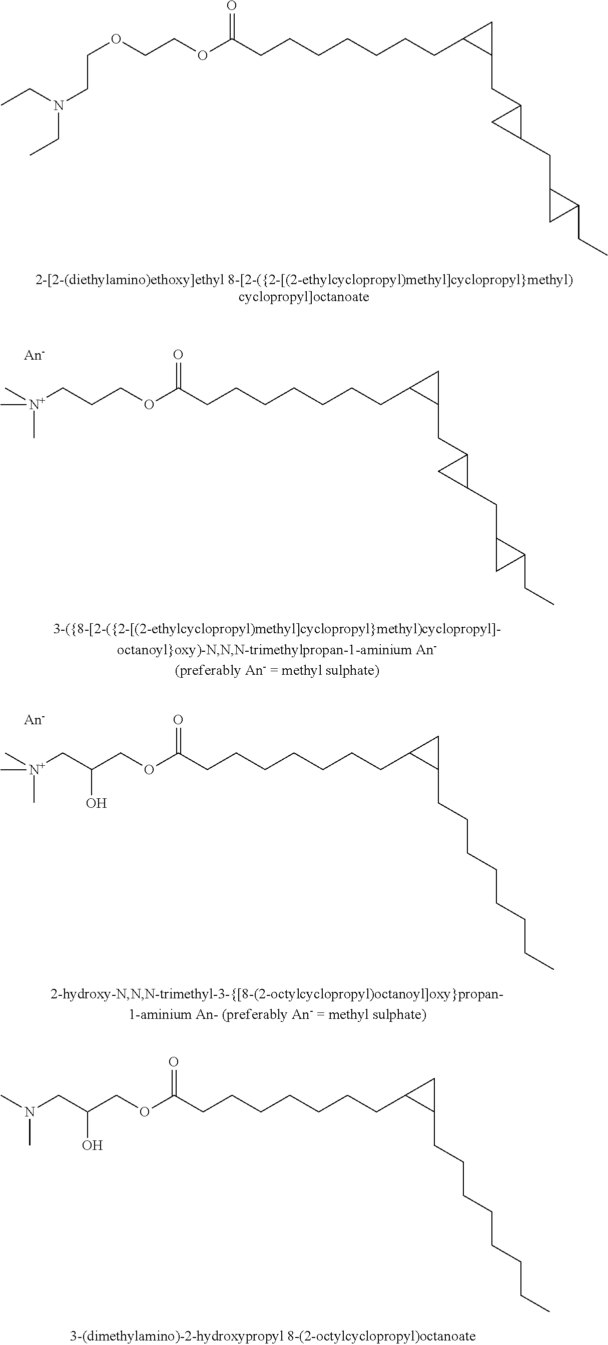

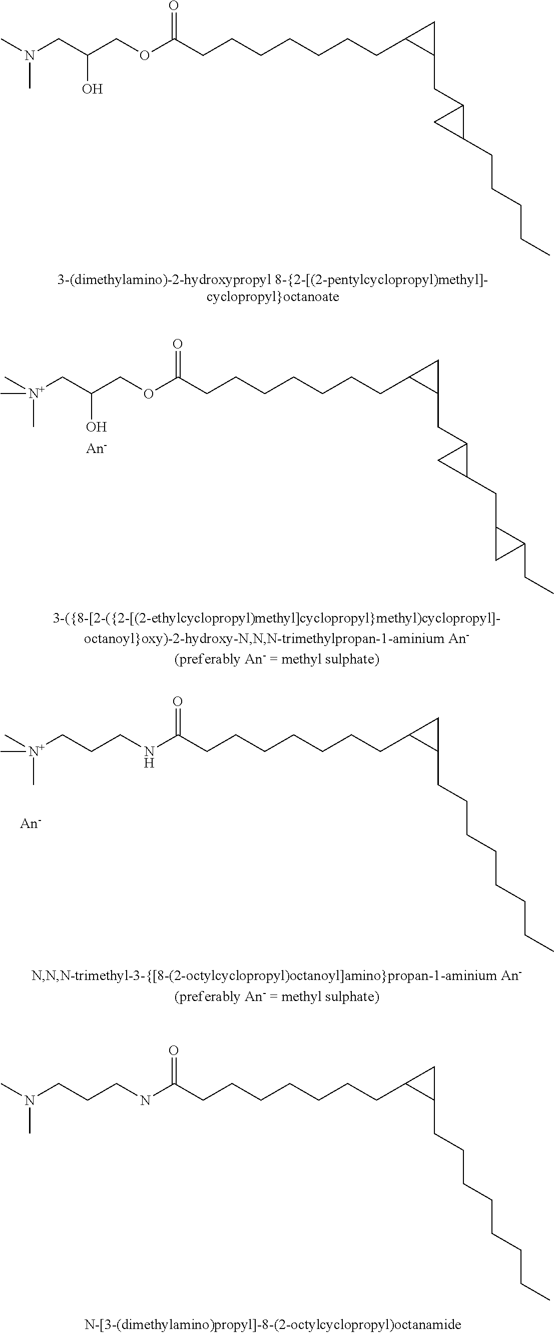

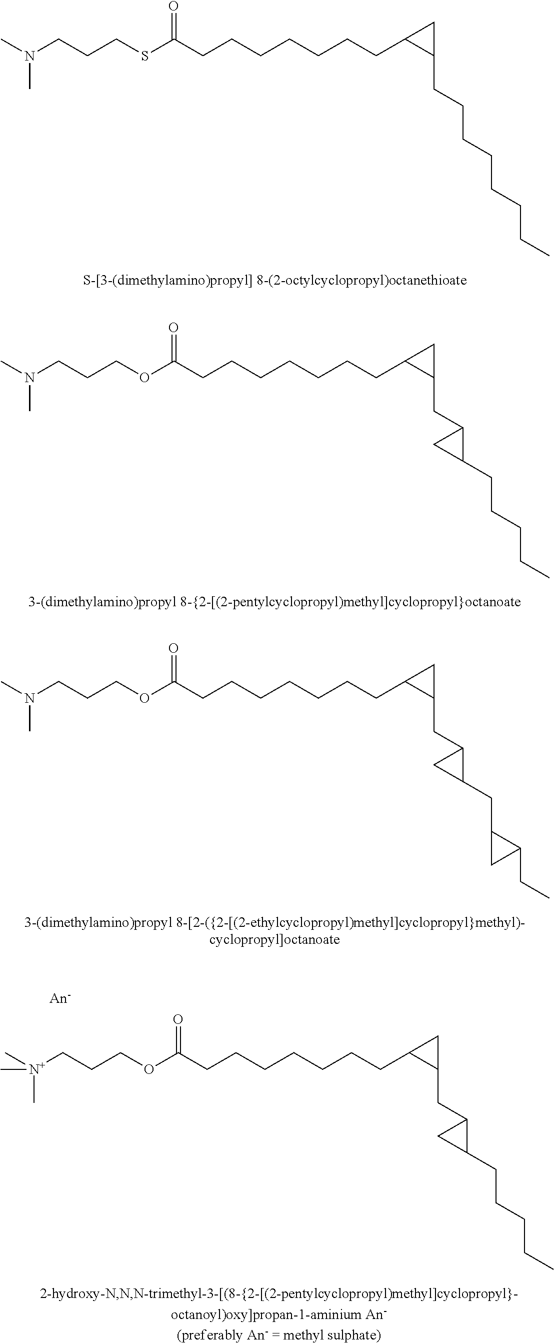

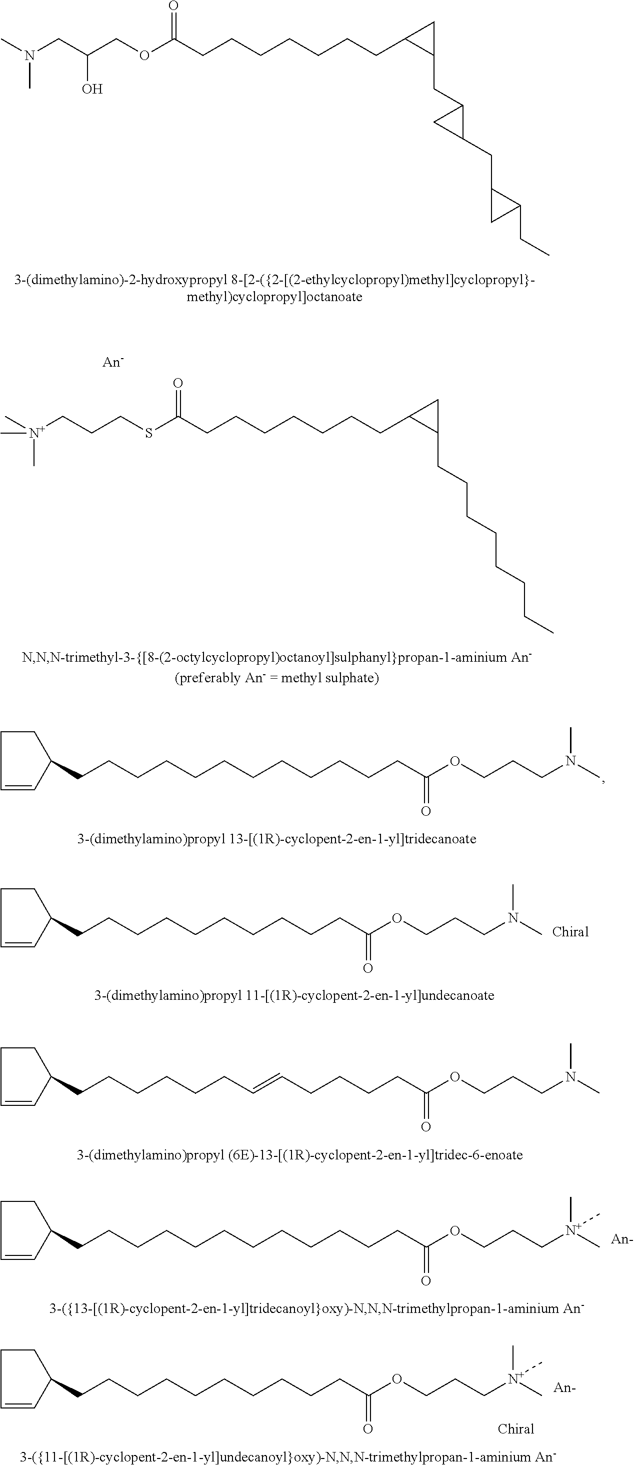

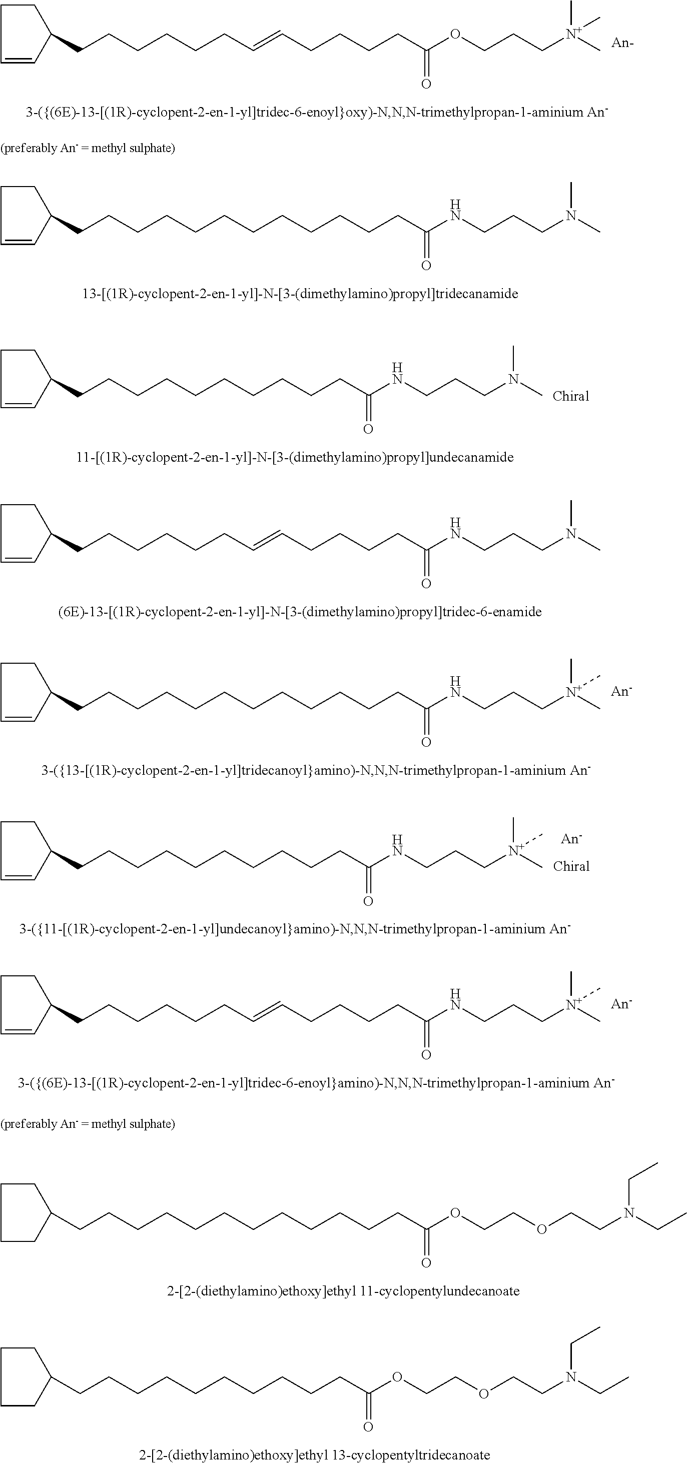

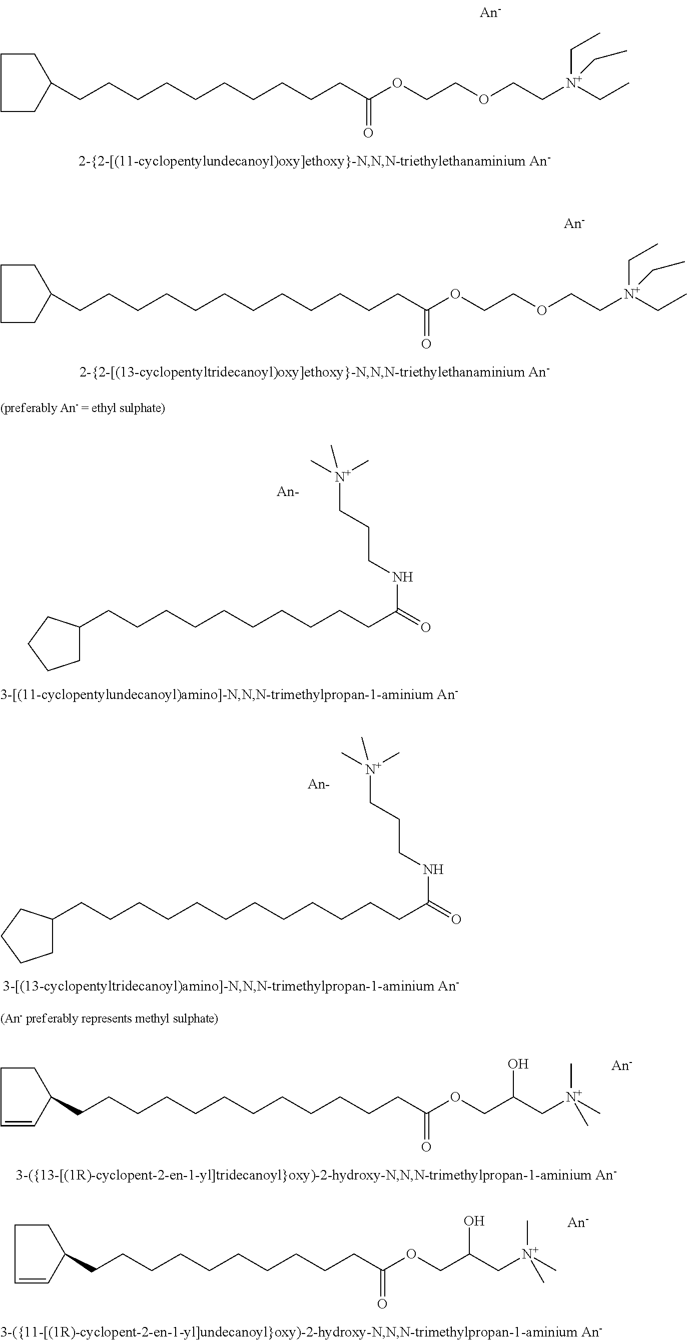

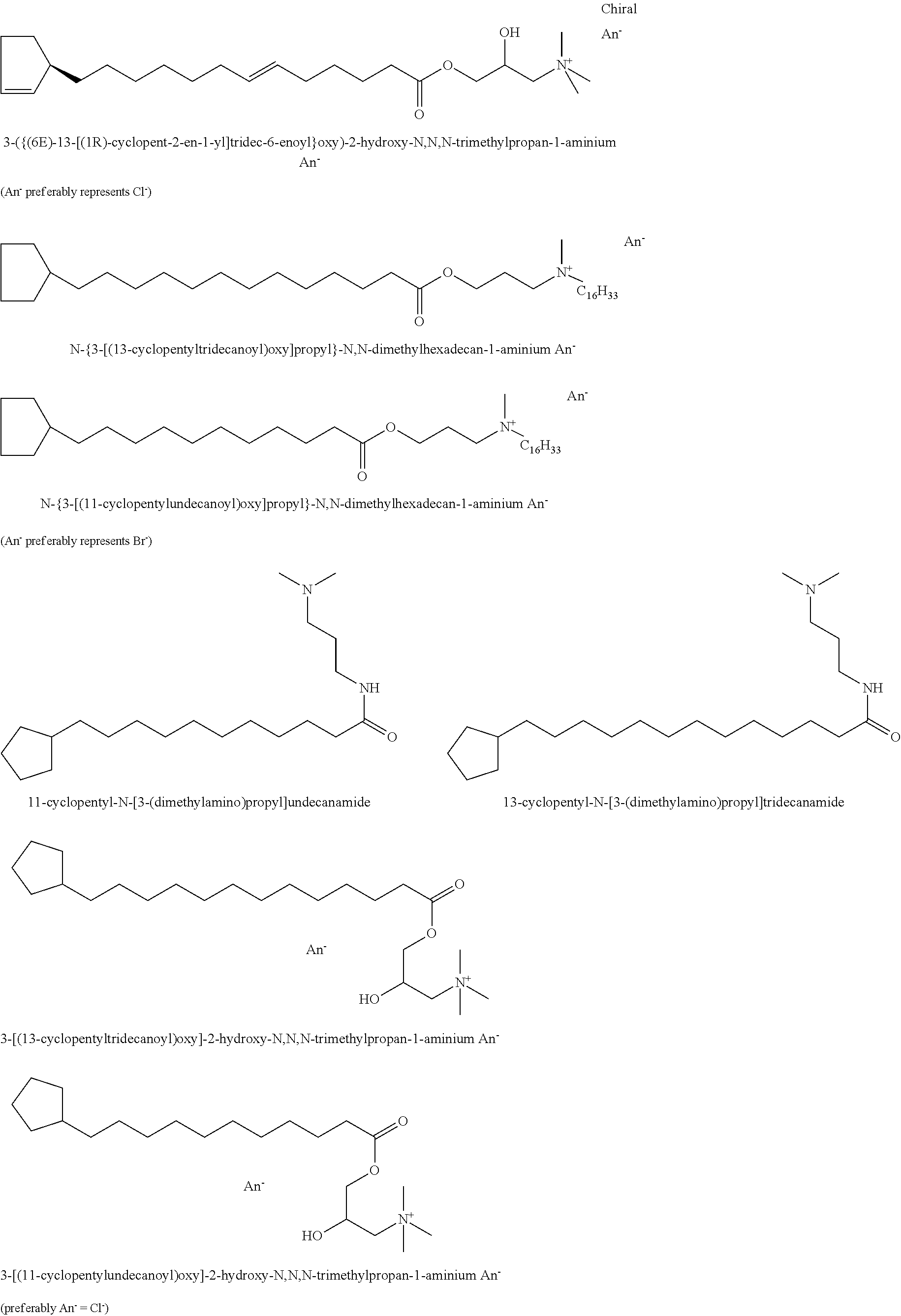

8. A cosmetic or pharmaceutical composition comprising, in a physiologically acceptable medium, at least one compound of formula (I) or (II) according to claim 1.

9. The composition according to claim 8, in which the amount of compound (I) or (II), alone or as a mixture, present in the composition is between 0.01 and 50% by weight, with respect to the total weight of the composition.

10. The composition according to claim 8, in which the physiologically acceptable medium comprises at least one standard cosmetic ingredient chosen from C₁-C₄₀ alcohols, carbon-comprising oils, water, C₈-C₄₀ esters, C₈-C₄₀ acids, nonionic surfactants, cationic surfactants, anionic surfactants, amphoteric surfactants, zwitterionic surfactants, propellants, sunscreens, moisturizing agents, antidandruff agents, antioxidants, reducing agents, oxidation bases, couplers, oxidizing agents, direct dyes, hair-straightening agents, pearlescent and opacifying agents, plasticizing or coalescence agents, hydroxy acids, pigments, fillers, silicones, organic solvents, polymeric or nonpolymeric thickeners, emulsifiers or polymers.

11. The composition according to claim 8, which is provided in the form of a product for caring for, cleaning and/or making up the skin of the body or of the face, the lips, the eyebrows, the eyelashes, the nails and the hair, of an antisun or self-tanning product, of a body hygiene product or of a hair product, in particular for caring for, cleaning, styling, shaping or dyeing the hair.

12. The composition according to claim 8, which is provided in the form of a hair composition for the care and the cosmetic treatment, in particular the protection, of the hair, especially weakened and/or damaged hair, for example hair weakened and/or damaged by chemical or mechanical treatments.

13. (canceled)

14. A method for the cosmetic treatment of keratinous substances comprising the application, to the said substances, of a cosmetic composition as defined in claim 8.

15. The method according to claim 14, wherein said method is a cosmetic treatment method for the conditioning of the hair, in particular for providing it with suppleness, disentangling, smoothing and/or the ability to be combed or for improving the suppleness, the disentangling, the smoothing and/or the ability to be combed thereof.
